(19) United States
(12) Patent Application Publication
(10) Pub. No.: US 2004/0143283 A1
(43) Pub. Date: Jul. 22, 2004

(54) Inflation adaptor and method of use

(76) Inventors: Scott McGill, Redwood City, CA (US); George Tsai, Sunnyvale, CA (US); Pablo Acosta, Newark, CA (US); Celso J. Bagaoisan, Union City, CA (US); Allan Steingisser, Beverly, MA (US); Morgan Matthew House, Portsmouth, NH (US); Thierry H. Benjamin, Lowell, MA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/348,046

(22) Filed: Jan. 17, 2003

(51) Int. Cl. A61M 29/00

(52) U.S. Cl. 606/192

(57) ABSTRACT

An inflation adaptor is provided to move a wire within a hollow tubular body, the movement of the wire enabling actuation of an expandable member at the distal end of the hollow tubular body. The expandable member can be a balloon, filter or similar device. The adaptor generally includes a clamping mechanism which clamps the hollow tubular body and wire within the adaptor, and also includes a drive mechanism for moving the wire within the hollow tubular body. An actuator, such as a rotatable knob, is provided to operate both the clamping mechanism and the drive mechanism. The adaptor may also include a fluid delivery and expansion system within a housing when the expandable member is a balloon, wherein movement of the actuator delivers a predetermined amount to the balloon while still operating the clamping mechanism and the drive mechanism.

### BACKGROUND OF THE INVENTION

1. Field of the Invention

The present invention relates in one embodiment to an apparatus and method of properly inflating and deflating a surgical balloon and, in particular to an inflation adaptor and method of using the same in a convenient and precise manner to properly inflate and deflate a surgical balloon.

2. Description of the Related Art

Human blood vessels often become occluded or completely blocked by plaque, thrombi, emboli or other substances, which reduce the blood carrying capacity of the vessel. Should the blockage occur at a critical location in the circulation, serious and permanent injury, or death, can occur. To prevent this, some form of medical intervention is usually performed when significant occlusion is detected, such as during an acute myocardial infarction (AMI).

Coronary heart disease is the leading cause of death in the United States and a common occurrence worldwide. Damage to or malfunction of the heart is caused by narrowing or blockage of the coronary arteries (atherosclerosis) that supply blood to the heart. The coronary arteries are first narrowed and may eventually be completely blocked by plaque, and may further be complicated by the formation of thrombi (blood clots) on the roughened surfaces of the plaques. AMI can result from atherosclerosis, especially from an occlusive or near occlusive thrombus overlying or adjacent to the atherosclerotic plaque, leading to death of portions of the heart muscle. Thrombi and emboli also often result from myocardial infarction, and these clots can block the coronary arteries, or can migrate further downstream, causing additional complications.

The carotid arteries are the main vessels which supply blood to the brain and face. The common carotid artery leads upwards from the aortic arch, branching into the internal carotid artery which feeds the brain, and the external carotid artery which feeds the head and face. The carotid arteries are first narrowed and may eventually be almost completely blocked by plaque, and may further be complicated by the formation of thrombi (blood clots) on the roughened surfaces of the plaques. Narrowing or blockage of the carotid arteries is often untreatable and can result in devastating physical and cognitive debilitation, and even death.

Various types of intervention techniques have been developed which facilitate the reduction or removal of the blockage in the blood vessel, allowing increased blood flow through the vessel. One technique for treating stenosis or occlusion of a blood vessel is balloon angioplasty. A balloon catheter is inserted into the narrowed or blocked area, and the balloon is inflated to expand the constricted area. In many cases, near normal blood flow is restored. It can be difficult, however, to treat plaque deposits and thrombi in the coronary arteries, because the coronary arteries are small, which makes accessing them with commonly used catheters difficult. Other types of intervention include atherectomy, deployment of stents, introduction of specific medication by infusion, and bypass surgery.

Furthermore, the fear of dislodging an embolus from an ulcerative plaque and the severe resulting consequences has prevented the widespread use of angioplasty in the carotid arteries. Because of the potential complications, the options for minimally invasive treatment of the carotid arteries are severely limited.

Carotid endarterectomy is another type of intervention for removal of blockages from the carotid arteries. In endarterectomy, the carotid bifurcation is exposed through an incision in the neck of the patient. Clamps are placed on either side of the occlusion to isolate it, and an incision made to open the artery. The occlusion is removed, the isolated area irrigated and aspirated, and the artery sutured closed. The clamps are removed to reestablish blood flow through the artery. In carotid endarterectomy, the emboli and debris are contained and directed by activating and deactivating the clamps. For example, after the clamps are in place, one on the common carotid artery and one on the internal carotid artery, the particles are contained between the two clamps. After the occlusion is removed, the clamp on the common carotid artery is opened, allowing blood to flow into the previously isolated area toward the clamp on the internal carotid. This blood flow is then aspirated through an external aspiration tube. The common carotid artery is then reclamped, and the clamp on the internal carotid opened. This causes blood to flow into the previously isolated area toward the clamp on the common carotid artery. The flow is then aspirated. The clamp on the internal carotid artery is closed, and the artery is sutured closed. This method allows for the flushing of debris into the area where aspiration occurs.

Alternatively, this method of clamping and unclamping the carotid arteries can be done after the incision in the artery is sutured closed. Using this method, it is hoped that any particles in the internal carotid artery will be forced back to the common carotid artery, then into the external carotid area, where serious complications are unlikely to arise from emboli.

Carotid endarterectomy is not without the serious risk of embolization and stroke caused by particles of the blocking material and other debris moving downstream to the brain, however.

There is therefore a need for improved methods of treatment of occluded vessels which decrease the risks to the patient.

Surgical balloons are used for procedures such as percutaneous transluminal angioplasty for treatment of stenosis and for occluding blood vessels to prevent release of emboli into the bloodstream during such procedures. During this type of procedure, a guidewire is conventionally used to guide the insertion of the medical instrument, such as a balloon catheter, to the desired treatment site within a patient's vasculature. A hollow guidewire or guidewire catheter with a balloon at its distal tip has more recently been seen employed to occlude a vessel distal of the treatment site and prevent emboli that is broken off during the procedure from migrating downstream (“distal protection”). Distal protection devices can also employ filters that provide for either partial or total occlusion of the vessel.

Surgical balloons used for distal protection are often made of compliant material and increase in diameter with increasing inflation pressure until the balloon burst pressure is reached. In practice, occlusion balloons should be expanded to contact the blood vessel wall. Clinicians, however, often do not know exactly when the balloon has contacted the blood vessel walls, if uniform circumferential occlusion has been accomplished or whether the balloon has been overinflated.

Conventional surgical balloons are inflated with a syringe coupled to the proximal end of the catheter. The syringe, which is located external to the patient, typically has a fluid capacity of anywhere from 0.5 cc to 10 cc and the clinician uses the syringe to inflate the balloon. The clinician must have considerable patience, skill and concentration to accurately deliver a suitable volume of liquid, such as 0.05 cc, to properly inflate the balloon.

The clinician must also be extremely careful not to overinflate the balloon. Although a pressure gauge is provided on some syringes, the skill required to avoid overinflation is still beyond many clinicians because a very small movement of the syringe piston results in a relatively large injection of fluid. For example, if the clinician desires to deliver about 0.1 cc of fluid to the balloon from a conventional 10 cc syringe, the travel of the syringe piston is less than about 0.7 mm. Thus, it can be readily seen that the control of the syringe to this degree of precision is very difficult. Additionally, unlike therapeutic balloons (which require about 20 atmospheres pressure and can use syringes ranging between about 10 to 20 cc in fluid capacity), typical occlusion balloons require less than about 3 atmospheres pressure and require less than about 1 cc of fluid. Because occlusion balloons are inflated to relatively low pressures with small amounts of fluids, the clinician must be very careful when using a conventional syringe to inflate the balloon.

The risks of imprecision while inflating a surgical balloon with a conventional syringe are substantial. For example, overinflation of the occlusion balloon may cause it to rupture, releasing inflation media into the bloodstream (e.g., fluid, air, gas, etc.), and may possibly allow pieces of the balloon to enter the bloodstream. In addition, the balloon will fail to trap emboli. Overinflation of the balloon can also damage the healthy tissue adjacent the vessel segment undergoing treatment, even if the balloon does not rupture. The radial expansion of the balloon can also cause undesirable pressure on the vessel wall, and longitudinal expansion of the balloon can create a shearing force which could lead to vessel trauma. Further, if the balloon is overinflated, it may experience a decrease in fatigue strength. For example, if a surgical balloon is overinflated such that it is approximately two to three times its original working diameter, the balloon may experience a significant decrease in fatigue strength. Underinflation of the balloon also causes many difficulties and problems. An underinflated balloon, for example, may allow fluid to flow around the balloon and the balloon may fail to trap emboli or anchor the guidewire in the desired position.

It is also very difficult for the clinician to deliver the desired volume of fluid and then maintain the syringe in a fixed location such that the volume of fluid does not subsequently change. For example, once the clinician has depressed the plunger of the syringe a desired amount to properly inflate the balloon, the clinician must hold the plunger in that position until the pressure equalizes and/or it is desired to deflate the balloon. As discussed above, even small movements of the syringe plunger may cause overinflation or underinflation of the balloon. Thus, the clinician must be very careful not to allow the plunger to move even a very small distance after the fluid is delivered because that may affect the amount of fluid delivered by the syringe.

In addition to the problems of overinflation, another problem exists when inflating occlusion balloons. As discussed above, even though the pressure required to inflate the occlusion balloon is generally less than 3 atmospheres, the pressure caused by a conventional inflation syringe causes an immediate build up of pressure near the syringe. The build up of pressure can reach magnitudes of 400 psi. The high pressure caused by conventional syringes often causes leaks in the system and it may damage the balloon. Additionally, this high pressure makes it very difficult for the clinician to properly inflate the balloon to the desired size and pressure.

Inflation adaptors already exist and are described in assignee's U.S. Pat. No. 6,050,972, the disclosure of which is hereby incorporated by reference. Improved inflation adaptors are desired to resolve problems as discussed above.

### SUMMARY OF THE INVENTION

An inflation adaptor is provided in accordance with certain embodiments of the present invention. A method of using the adaptor is also provided.

In one embodiment, an adaptor for controlling actuation of an expandable device is provided. The adaptor comprises a housing with a retaining portion which operates to releasably retain a section of a hollow tubular body therein, the expandable device being disposed at a distal end of the hollow tubular body. An actuator is mounted on the housing, wherein movement of the actuator clamps the tubular body within the retaining portion of the housing and also moves a wire at least partially within the hollow tubular body from a first position to a second position. The movement of the wire between the first and second positions enables actuation of the expandable device. In one embodiment, the expandable device is a balloon, and the actuator moves a wire within a lumen of the tubular body to enable fluid to pass through an inflation port in the hollow tubular body to inflate the balloon. In another embodiment, the expandable device is a filter, and the actuator actuates the filter by moving a pull wire connected to the filter and extending through the lumen of the hollow tubular body.

In another embodiment, an adaptor is provided for controlling actuation of an expandable device, the expandable device being disposed on a distal end of a hollow tubular body. The hollow tubular body surrounds a wire at least at a proximal end of the hollow tubular body, and the wire is moveable within the hollow tubular body from a first position to a second position, the movement of the wire between the first and second positions enabling actuation of the expandable device. The adaptor of this embodiment comprises a housing having a first panel and a second panel disposed therein. The first panel and second panel define a channel there between, the channel being adapted to receive a portion of the hollow tubular body and the wire. The second panel is moveable toward and away from the first panel. A first sliding pad and a second sliding pad are positioned within openings of the first and second panels, respectively, and are adapted to receive a portion of the wire there between. The sliding pads are cooperatively slidable within the openings of the first and second panels in a longitudinal direction. An actuator is operatively connected to at least the second panel and at least the second sliding pad. Movement of the actuator causes the second panel to move relatively toward the first panel and also causes the sliding pads to move within the openings of the first panel and second panel. The actuator moves from a first position wherein the first panel and second panel are spaced apart, to a second position wherein the first panel and second panel are pressed against each other in order to clamp the hollow tubular body there between. The actuator moves to a third position wherein the first sliding pad and second sliding pad move within the openings of the first and second panels to move the wire from its first position to its second position within the hollow tubular body.

In another embodiment, an integrated inflation adaptor is provided for inflating a balloon through an inflation port on a hollow tubular body. The adaptor comprises a housing with a retaining portion which operates to releasably retain a section of the hollow tubular body therein such that the inflation port is positioned within the retaining portion adjacent a fluid opening within the retaining portion. The retaining portion is also adapted to retain a wire moveably disposed within a proximal end of the hollow tubular body. An actuator is mounted on the housing, wherein the actuator is moveable from a first position to a second position to clamp the tubular body within the retaining portion of the housing, and is moveable from the second position to a third position to move the wire within the hollow tubular body in a distal to proximal direction across said inflation port. This thereby allows fluid flow from the fluid opening through the port.

In another embodiment, a method of manipulating a wire within a lumen of a hollow tubular body is provided. The method comprises positioning the hollow tubular body and wire within a retaining portion of an adaptor. The retaining portion includes a first panel and a second panel defining a channel there between for positioning of at least the tubular body, and first and second pads adapted to position the wire there between. An actuator on the adaptor is moved from a first position to a second position, the movement of the actuator causing the first panel and second panel to move relatively toward each other to clamp at least the hollow tubular body there between. When the hollow tubular body is clamped between the first panel and second panel, the first and second pads contact the wire. The actuator is moved from the second position to a third position, the movement of the actuator causing movement of the first and second pads in a direction parallel to a longitudinal axis of the hollow tubular body to cause corresponding movement of the wire relative to the hollow tubular body.

### BRIEF DESCRIPTION OF THE DRAWINGS

FIG. 1 is a side view of an occlusion balloon guidewire which can be used in accordance with embodiments of the present invention.

FIG. 2A is a partial cross-sectional view of a valve mechanism incorporated into the guidewire of FIG. 1.

FIG. 2B is an enlarged view of the valve mechanism of FIG. 2A, showing the valve mechanism in an open position (and a closed position shown in phantom).

FIG. 3 is a perspective view of an inflation adaptor in accordance with a first embodiment of the present invention.

FIG. 4 is a perspective view of the inflation adaptor of FIG. 3 without a cover.

FIG. 5 is an exploded assembly view of the inflation adaptor of FIG. 3.

FIG. 6 is a detailed perspective view of the clamping and drive assemblies of the inflation adaptor of FIG. 3.

FIG. 7 is a detailed perspective view of the drive assembly of the inflation adaptor of FIG. 3.

FIGS. 8A-B are detailed perspective views of the panels of the inflation adaptor of FIG. 3.

FIG. 8C is a detailed perspective view of the rear side of the panel shown in FIG. 8B.

FIGS. 9A-9F are perspective views of an assembly sequence for a sliding pad incorporated into the inflation adaptor of FIG. 3.

FIGS. 9G and 9H are side views of the sliding pad of FIGS. 9A-9F.

FIGS. 10A-10D are perspective views of an assembly sequence for another sliding pad incorporated into the inflation adaptor of FIG. 3.

FIG. 10E is a side view of the first sliding pad of FIGS. 10A-10D.

FIG. 11 is a perspective view of the inflation adaptor of FIG. 3, shown operably connected to an occlusion balloon guidewire deployed in a blood vessel.

FIGS. 12A and 12B are diagrams illustrating overdrive systems in accordance with embodiments of the present invention.

FIGS. 13A-13H are perspective views of an assembly sequence for an inflation adaptor according to one embodiment of the present invention.

FIGS. 14A and 14C-14E are perspective views of an assembly sequence for a panel incorporated into the adaptor of FIGS. 13A-13H.

FIG. 14B is a side view of the panel of FIG. 14A.

FIGS. 14F and 14G are side views of an assembled panel in accordance with FIGS. 14A-14E.

FIG. 15 is a perspective view of an inflation adaptor in accordance with a second embodiment of the present invention.

FIG. 16 is a perspective view of the inflation adaptor of FIG. 15 without a cover.

FIG. 17 is an exploded assembly view of the inflation adaptor of FIG. 15.

FIG. 18 is a detailed top view of the drive assembly of the inflation adaptor of FIG. 15.

FIG. 19 is a detailed bottom view of the drive assembly of the inflation adaptor of FIG. 15.

FIG. 20 is a side view of the inflation adaptor of FIG. 15, with a portion of the adaptor cut away.

FIG. 21 is a perspective view of the housing of the inflation adaptor of FIG. 15 partially assembled.

FIG. 22 is a top view of the inflation adaptor of FIG. 15.

FIG. 23 is a top view of the inflation adaptor of FIG. 15 in a first position, shown without a cover.

FIG. 24 is a top view of the inflation adaptor of FIG. 15 in a second position, shown without a cover.

FIG. 25 is a top view of the inflation adaptor of FIG. 15 in a third position, shown without a cover.

FIG. 26 is a perspective view of an inflation adaptor in accordance with a third embodiment of the present invention.

FIG. 27 is a perspective view of the inflation adaptor of FIG. 26 with the outer cover removed.

FIG. 28 is an enlarged top view of the sliding plate of the inflation adaptor of FIG. 26.

FIG. 29 is a perspective view of the inner panels of the inflation adaptor of FIG. 26.

It will be appreciated that the figures described herein are merely exemplifying, and that the figures may not necessarily be to scale.

### DETAILED DESCRIPTION OF THE PREFERRED EMBODIMENTS

One embodiment of the present invention is adapted for use in the treatment of a stenosis or an occlusion in a blood vessel in which the stenosis or occlusion has a length and a width or thickness which at least partially occludes the vessel's lumen. Thus, the method is effective in treating both partial and complete occlusions of blood vessels.

It is to be understood that “occlusion” as used herein with reference to a blood vessel is a broad term and is used in its ordinary sense and includes both complete and partial occlusions, stenoses, emboli, thrombi, plaque and any other substance which at least partially occludes the lumen of the blood vessel. The term “occlusive device” as used herein is a broad term and is used in its ordinary sense and includes balloons, filters and other devices which are used to partially or completely occlude the blood vessel prior to, during or after performing therapy on the occlusion. It will be appreciated that even when a filter is used, the filter may be partially or completely occlusive.

The methods of the present invention are particularly suited for use in removal of occlusions from saphenous vein grafts, coronary and carotid arteries, and other vessels having similar pressures and flow. It will be appreciated that the methods described herein are not limited to the particular sequences described, and therefore, other sequences may be used as desired.

### I. Overview of an Occlusion Balloon Guidewire

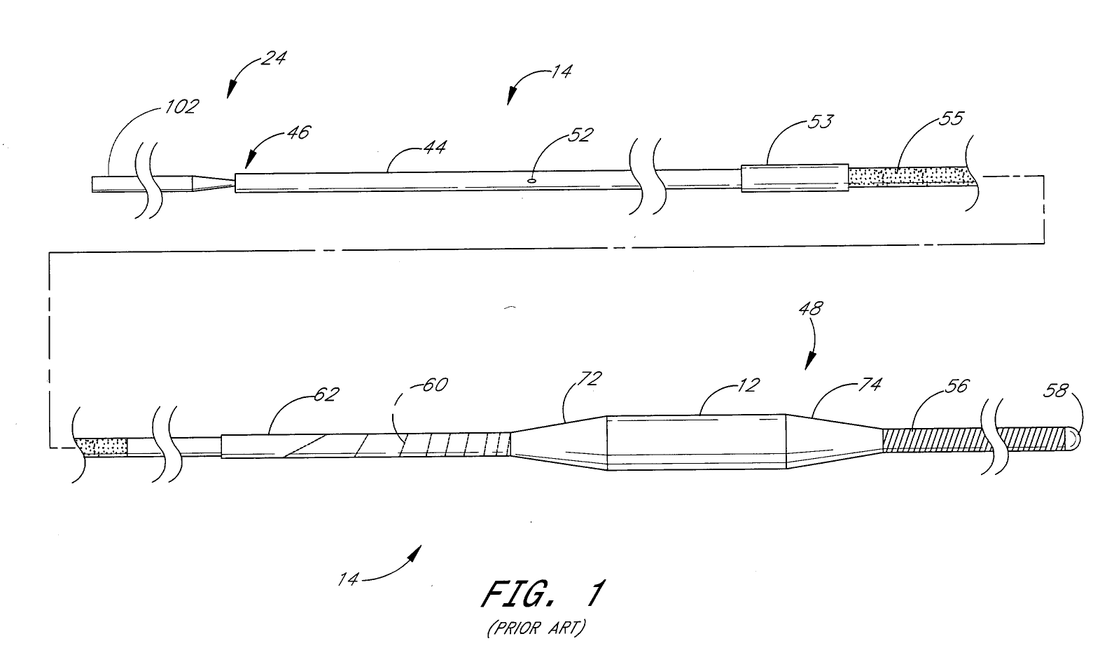

FIG. 1 illustrates one embodiment an occlusive device that can be used in combination with the adaptors described below. In the embodiment shown, the occlusive device is an occlusion balloon guidewire. The occlusion balloon guidewire 14 shown performs the function of occluding a vessel and allowing for the slidable insertion or advancement of various other catheters and devices. The term “guidewire” or “occlusion balloon guidewire” as used herein is intended to include both guidewires and catheters with these desired characteristics. One suitable guidewire system is available from Medtronic AVE under the name GUARDWIRE PLUS™.

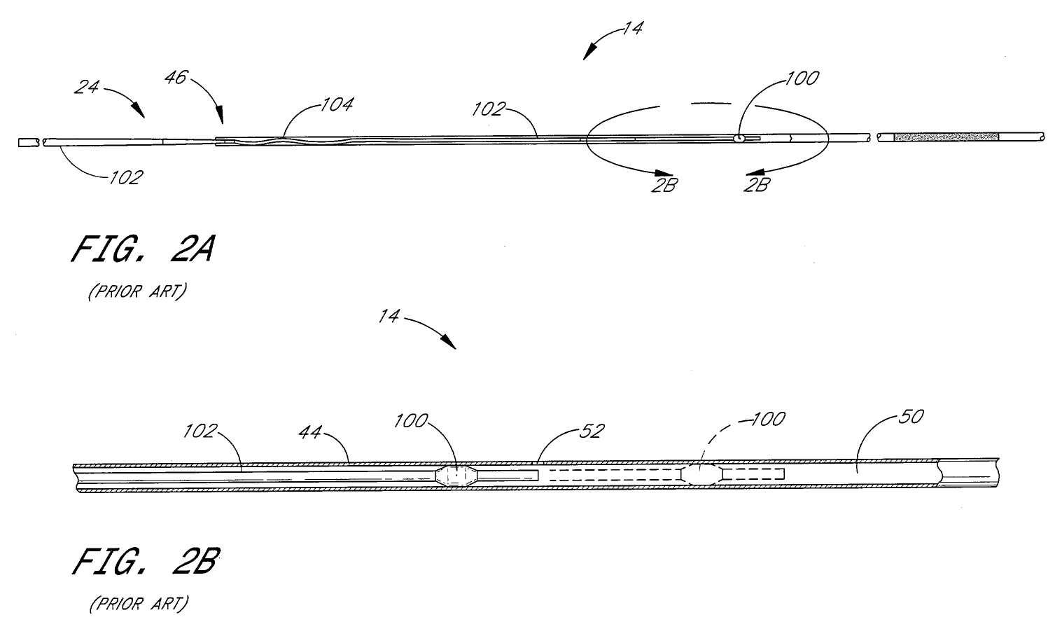

As shown in FIG. 1, an occlusion balloon guidewire 14 generally comprises an elongate flexible tubular body 44 extending between a proximal control end 46, corresponding to a proximal section of the tubular body 44, and a distal functional end 48, corresponding to a distal section of tubular body 44. Tubular body 44 has a central lumen 50, shown in FIG. 2B, which extends between the proximal and distal ends. An inflation port 52, shown also in FIGS. 2A and 2B described below, is provided on tubular body 44 near the proximal end 46. Inflation port 52 is in fluid communication with lumen 50 such that fluid passing through inflation port 52 into or out of the lumen 50 may be used to inflate or deflate an inflatable balloon 12 in communication with lumen 50.

A wire 102, as described below, is inserted into the proximal end 46 of the tubular body 44 to control inflation of a balloon 12 mounted on the distal end of the tubular body through inflation port 52. A marker 53, which may be made of gold, is placed over the tubular body 44 distal to the inflation port 52. Distal to the marker 53, a nonuniform coating 55 of polymer material, for example polytetrafluoroethylene (PTFE), is applied to the tubular body 44, terminating proximal to a shrink tubing 62. The shrink tubing 62 extends up to and within the balloon 12, and covers spiral cuts 60 formed in the tubular body 44. These spiral cuts 60 extend to a location between the proximal and distal ends of the balloon 12, and distal to the shrink tubing 62, such that fluid delivered through the lumen 50 enters the balloon 12 through the turns of the cuts 60. Adhesive tapers 72 and 74 extend from the proximal and distal ends of the balloon 12, respectively. The proximal taper 72 extends from the proximal end of the balloon to the shrink tubing 62 on the tubular body 44, while the distal taper 74 extends to coils 56 extending from the distal end 48 of the tubular body 44. The coils 56 terminate in a distal ball 58.

Other details regarding construction of the balloon guidewire described above as well as similar devices may be found in assignee's U.S. Pat. No. 6,068,623, U.S. Pat. No. 6,228,072, and copending applications entitled FLEXIBLE CATHETER, application Ser. No. 09/253,591, filed Feb. 22, 1999, and FLEXIBLE CATHETER WITH BALLOON SEAL BANDS, application Ser. No. 09/653,217, filed Aug. 31, 2000, all of which are hereby incorporated by reference in their entirety.

As shown in FIGS. 2A and 2B, the wire 102 is inserted into the lumen 50 of the hollow tubular body 44 and has a proximal end that is positioned outside of the hollow tubular body proximal to the proximal end 46. A movable sealer portion 100 is attached at a distal end of the wire 102 and is positioned within the inflation lumen 50 of the guidewire 14. In one embodiment, the wire 102 includes a zig-zag portion 104, which may be formed integrally or separate from the wire 102, the zig-zag portion 104 being proximal to the sealer portion 100 and providing a retention force to the wire 102 due to frictional engagement with the walls of the lumen 50. The sealer portion 100 forms a fluid tight seal with the inflation lumen 50 by firmly contacting the entire circumference of a section of the inflation lumen 50.

As shown in FIGS. 2A and 2B, the combination of the wire 102, the tubular body 44 having lumen 50, the inflation port 52 and the sealer portion 100 together form one embodiment of a valve mechanism 24. The sealer portion 100 may be positioned proximally of the inflation port 52 on the guidewire as shown in FIG. 2B, to establish an unrestricted fluid pathway between the inflation port 52 and the inflatable balloon 12 on the distal end. In this configuration, the valve mechanism 24 is “open.” As desired, the clinician may move the sealer portion 100 to a position at or distal of the inflation port 52, as shown in phantom in FIG. 2B, thereby preventing any fluid from being introduced into or withdrawn from the lumen 50 via the inflation port 52. In this configuration, the valve mechanism 24 is “closed.” The valve mechanism 24 in the embodiment shown is considered “low profile” because the wire 102 is no larger in cross-sectional diameter than the guidewire 14 itself. Further details of these features and other assemblies may be found in assignee's U.S. Pat. No. 6,050,972, the entirety of which is hereby incorporated by reference.

The occlusive device described above advantageously enables an exchange of catheters over the guidewire while the balloon is inflated, for example, to isolate particles within a blood vessel. For example, a therapy catheter such as a PTCA or stent delivery catheter can be delivered over the guidewire to perform treatment, and then be exchanged with an aspiration catheter to remove particles from the vessel. Further details of this exchange and various treatment procedures are described in assignee's copending application entitled EXCHANGE METHOD FOR EMBOLI CONTAINMENT, Ser. No. 09/049,712, filed Mar. 27, 1998 and in U.S. Pat. No. 6,135,991, the entirety of both of which are hereby incorporated by reference. It will also be appreciated that other occlusive devices may be used, such as pull wire or core wire filter devices, examples of which are described in assignee's U.S. Pat. No. 6,312,407 and U.S. application Ser. No. 10/099,399, filed Mar. 15, 2002, the entirety of each of which is hereby incorporated by reference.

### II. Inflation Adaptors

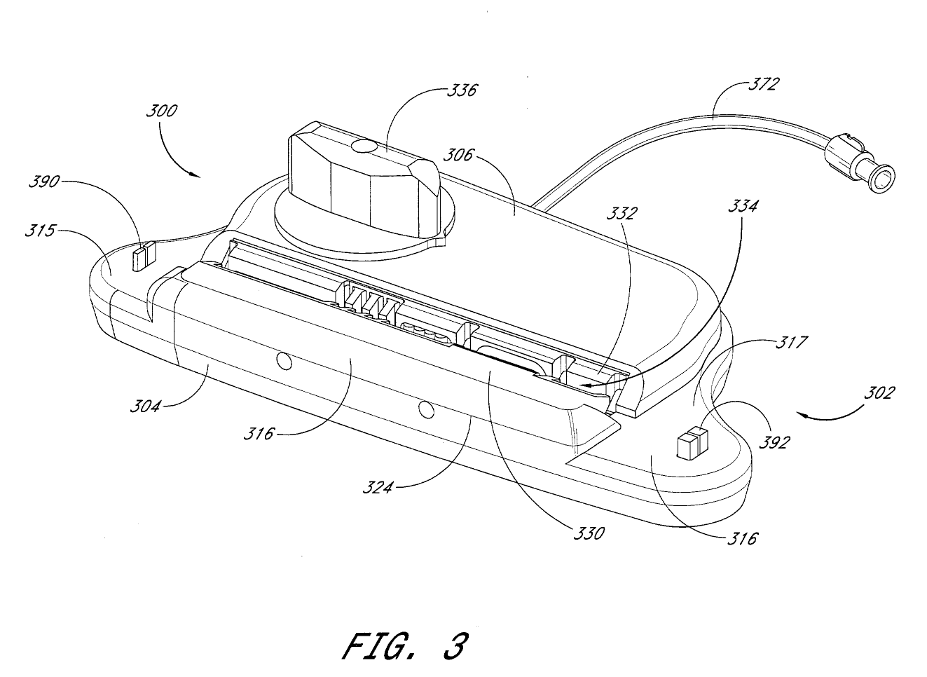

FIGS. 3-29 illustrate three embodiments of adaptors that can be used to operate the occlusive device described above. In particular, each of these adaptors can be used to operate the valve mechanism 24 of the guidewire 14 described above, and move the wire 102 longitudinally within the lumen 50 of the guidewire 14. As described further below, the guidewire 14 is releasably placed within a retaining portion of the adaptor. The adaptor includes an actuator that moves the wire 102 proximally such that the sealer portion 100 is proximal of the inflation port 52 and the valve mechanism 24 is in the open position. While in the open position, a fluid pathway can be established through the adaptor, into inflation port 52 and through lumen 50 to inflate the balloon 12. After balloon inflation is completed, the adaptor can be used to move the sealer portion 100 distally of the inflation port 52 such that the valve mechanism 24 is in the closed position. While in the closed position, the balloon 12 is maintained in its inflated state.

With the balloon 12 inflated and the valve mechanism 24 closed, the adaptor can be removed, and various treatment catheters can be delivered and exchanged over the guidewire 14 while the balloon 12 remains inflated. After treatment is completed, the adaptor can be reattached to the guidewire 14 to again move the sealer portion 100 proximal of the inflation port 52, such that fluid can be drawn out of the balloon 12, through lumen 50 and out of inflation port 52, to deflate the balloon 12.

It will be appreciated that the adaptors described herein have applicability not only to the balloon devices described above, but also to pull wire or core wire filter devices, and to any device having an inner wire that is moveable relative to an outer tube. In particular, although these adaptors are exemplified with respect to the guidewire 14 described above, these adaptors have applicability to any expandable device being disposed on a distal end of a hollow tubular body, the hollow tubular body surrounding an elongate member such as a wire at least at a proximal end thereof, and wherein the wire is moveable within the hollow tubular body from a first position to a second position. For these types of expandable devices, the movement of the wire between the first and second positions enables actuation of the expandable device. For example, in the embodiment describing an occlusion balloon guidewire above, the movement of the wire enables actuation by establishing a fluid pathway to the balloon 12 through the lumen 50 and the inflation port 52. In other embodiments, such as pull wire filter devices, the movement of the wire enables actuation simply because movement of the wire corresponds directly with the actuation of the filter.

A. First Embodiment

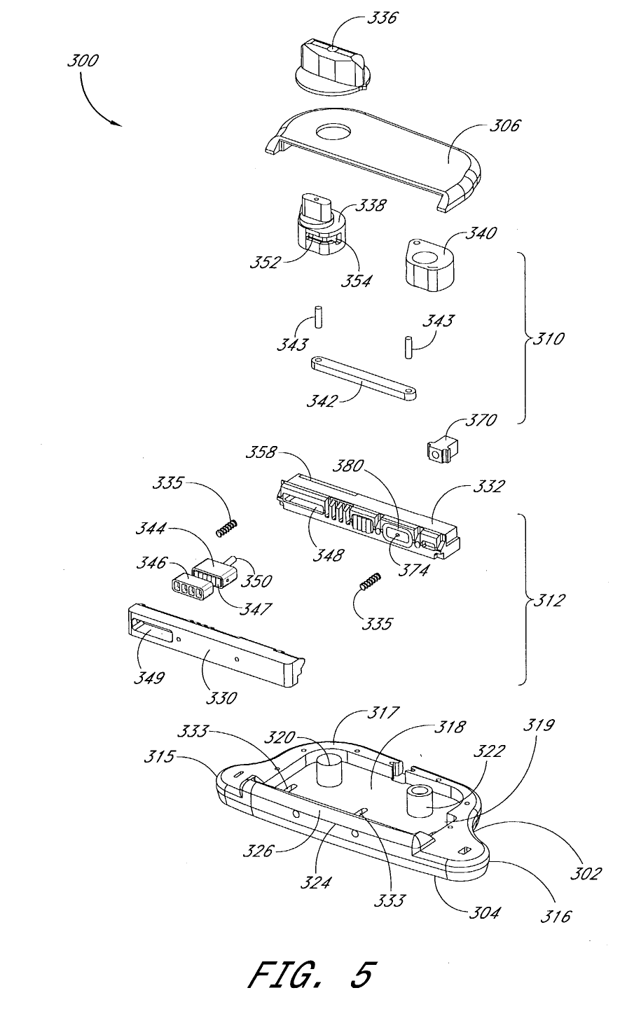

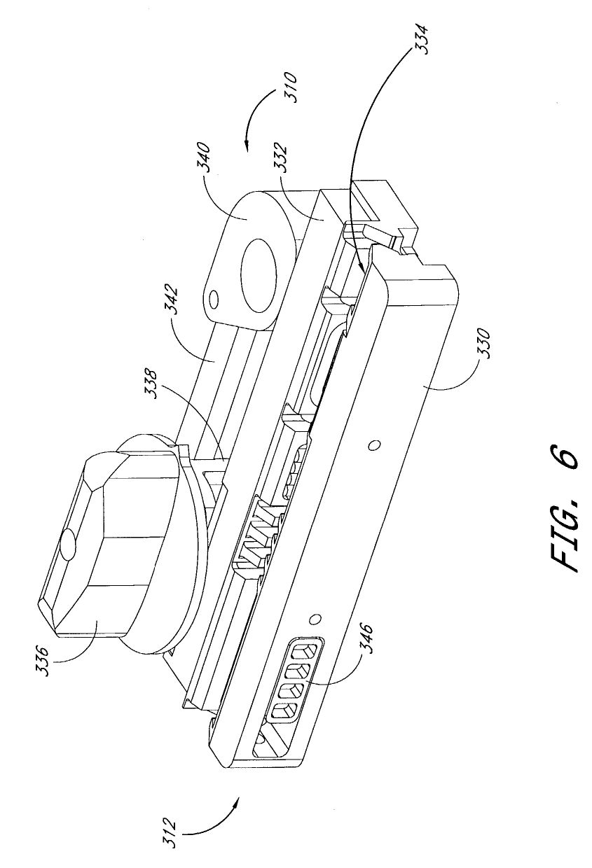

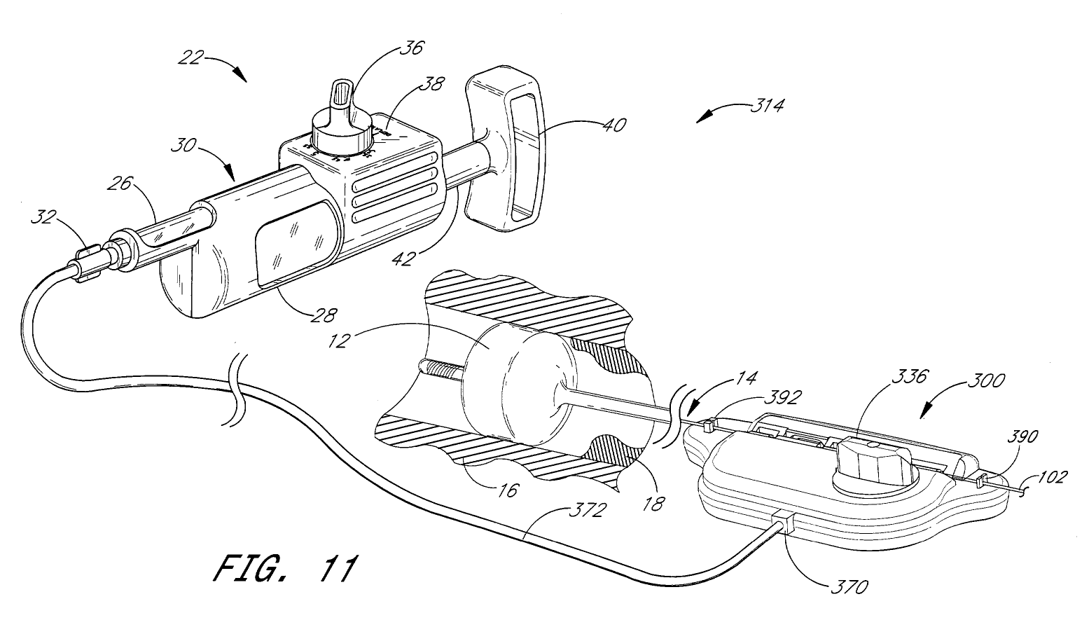

Referring to FIGS. 3-13B, there is illustrated a first embodiment of an inflation adaptor which may be used to inflate and to open and close the valve mechanism 24 depicted in FIGS. 2A-2B. Inflation adaptor 300, as shown in FIG. 3, comprises a housing 302 having a base 304 and a cover 306, which may be formed of metal, medical grade polycarbonate, or the like. However, as will be appreciated by those of skill in the art, a great many other materials may by used to form adaptor 300, including metals such as 300 series stainless steel and 400 series stainless steel, and polymeric materials such as acrylonitrile-butadiene-styrene (ABS), acrylics, and styrene-acrylonitriles. Furthermore, the base 304 and cover 306 may be manufactured in a variety of ways. For example, where polymeric materials are used, it is preferable to use a mold to manufacture the base 304 and the cover 306. Moreover, in some embodiments, more than one molded piece may be used to form base 304 and cover 306, with the various pieces being joined together by bonding or mechanical means to form either base 304 or cover 306. Alternately, as is known in the art, base 304 and cover 306 can be formed through machining processes performed on larger blocks of the raw materials. Adaptor 300 also includes a drive system 310 (see, e.g., FIGS. 5-7), and a clamping system 312 (see, e.g., FIGS. 5-6), and can be incorporated into a fluid delivery system 314 (see, e.g., FIG. 11), as will be described hereinafter.

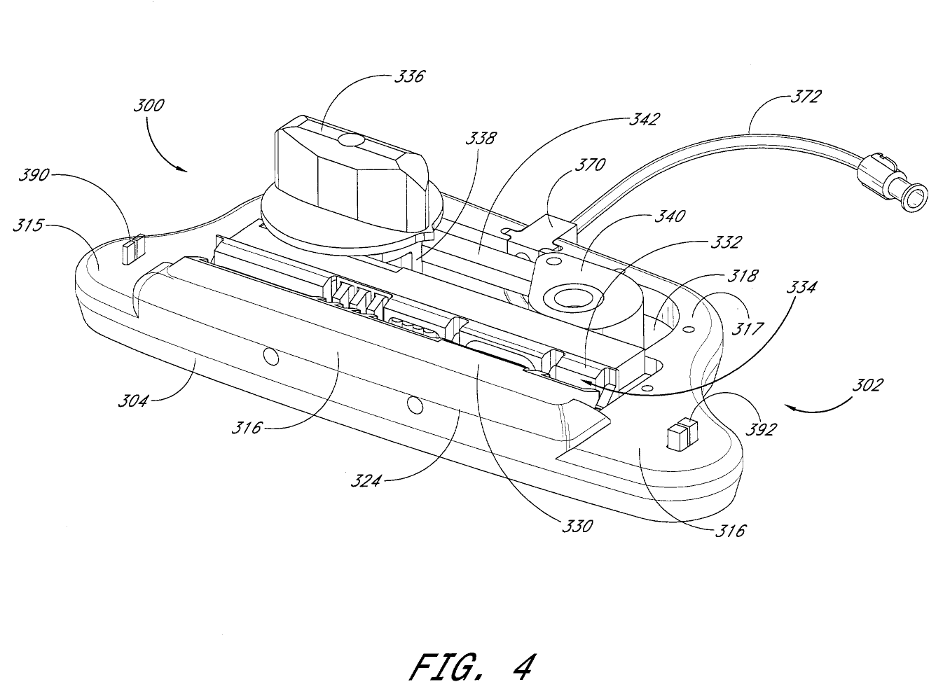

Base 304 in one embodiment has an asymmetric shape, as shown in FIGS. 3-4. Base 304 as illustrated has a proximal protrusion 315 at a proximal end of the adaptor and a distal protrusion 316 at a distal end of the adaptor, both of which extend from the horizontal main body of base 304. The base in one embodiment is about 0.75 in. high, about 3 in. wide, and about 4.5 in. in length across the main body and about 6.5 in. in length across the protrusions 315 and 316. The dimensions of the base are exemplifying, and it is envisioned that the dimensions may vary. The low profile and asymmetrical shape provide improved stability of the adaptor 300 during use. The asymmetric shape also ensures proper loading of the guidewire 14 as into adaptor 300 as will be described further below.

As shown in FIG. 5, base 304 includes an upper surface 317 having a central recess 318 configured to further support the drive system 310 and clamping system 312, as described below. Cover 306 may be permanently fixed to base 304, enclosing the drive system 310 and clamping system 312. For example, a plurality of screws may secure the base 304 and cover 306 to one another. Alternatively, cover 306 may be releasably secured to base 304 by a pair of hinges positioned on one of the lateral edges of base 304 and cover 306, such that base 304 and cover 306 may be separated or joined in a clam shell manner.

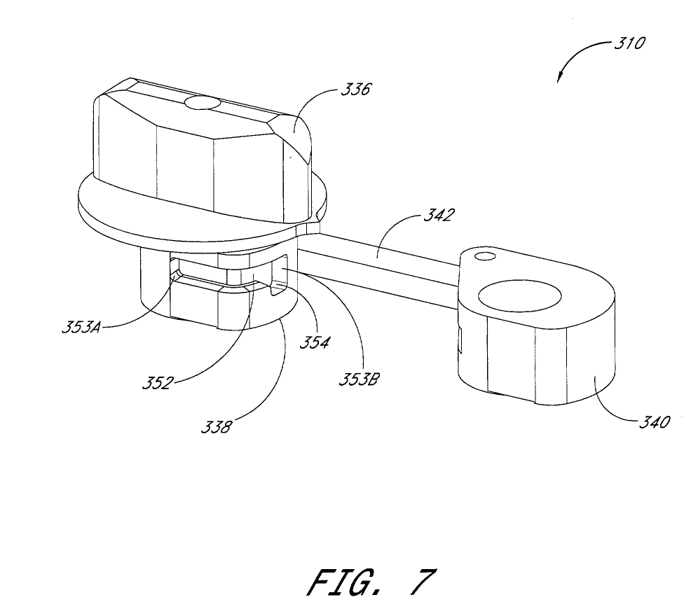

Central recess 318 includes at least two vertical cylindrical protrusions 320, 322, for supporting the drive system 310, shown also in FIG. 7. The protrusions 320, 322 are axles for the drive system 310, described below. The base 304 includes a horizontal projection 324 which extends along a majority of the length of one side of the recess 318, between the protrusions 315 and 316, forming a support wall 326 for supporting a panel 330. Panel 330 may be permanently affixed or press fit to support wall 326 when the adaptor is assembled as shown in FIG. 3. Another panel 332 is disposed across from panel 330 in recess 318 (see FIG. 4). As described below, panel 332 is moveable toward and away from mating panel 330.

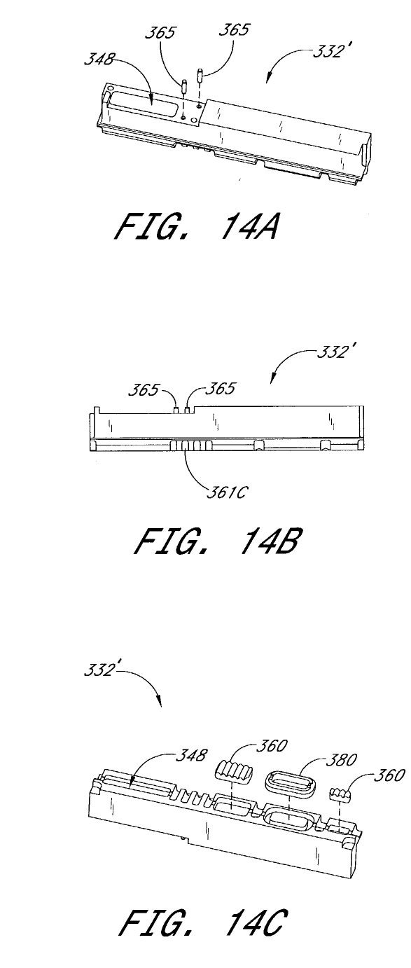

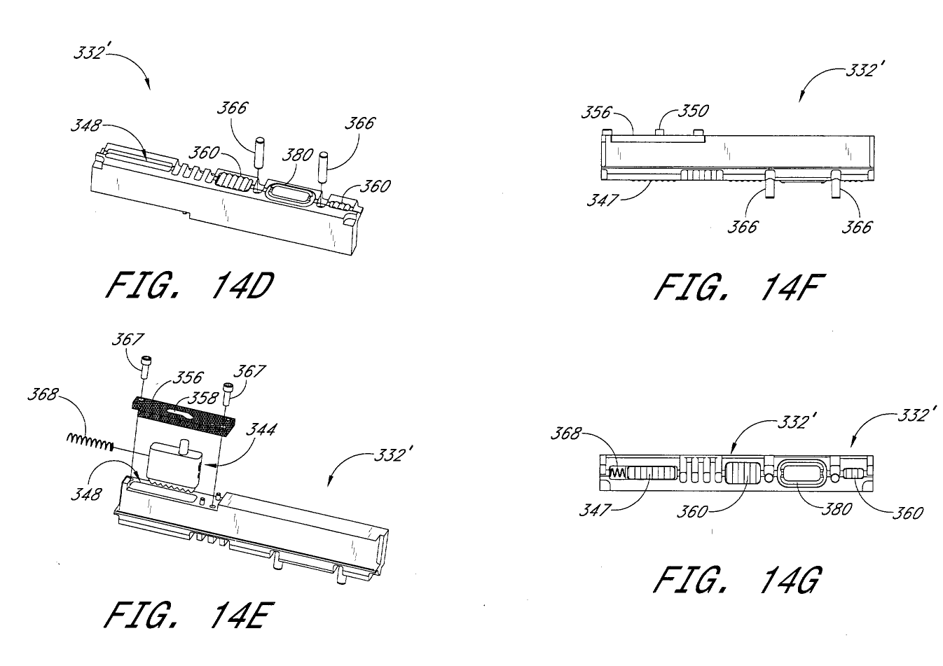

Together panels 330 and 332 comprise clamping system 312. As shown in FIG. 5, recess 318 includes an extension 319 toward the distal end of the adaptor to receive the distal ends of the panels 330 and 332. Recess 318 may also include tracks 333 with ball bearings and/or guiding pins (not shown, but see FIGS. 14A-14G illustrating guide pins 366) to guide the movement of panel 332 toward and away from panel 330. When the adaptor is assembled as shown in FIG. 3, a channel 334 is formed between the base 304 and cover 306, and panels 330, 332, for receiving the guidewire 14. Springs 335, shown in FIG. 5, may be positioned between panels 330 and 332 to bias panel 332 away from panel 330.

An actuator 336 is positioned on the external surface of cover 306. In the embodiment illustrated in FIGS. 5-7, actuator 336 controls and is operably connected to a first cam 338 and a second cam 340 mounted on protrusions 320 and 322, respectively. The cams 338 and 340 as illustrated are connected by a link 342 using pins 343, to form drive system 310. The actuator 336 may be a knob, which may be rotated about 30-360 degrees, more preferably about 90-100 degrees. The rotation may be clockwise or counterclockwise, or both. Although actuator 336 has been described as rotating, it is also envisioned that actuator 336 may slide or move in other ways. Cams 338, 340 in one embodiment have a lobe shape for driving the clamping system 312.

As shown in FIG. 6, cams 338 and 340 provide a uniform clamping strength whereby turning actuator 336 causes panel 332 to move toward panel 330. During a first movement of the actuator 336, panel 332 slides toward panel 330 when cams 338 and 340 rotate in unison in contact with panel 332. The lobe shape of cams 338, 340 applies a force upon contact with panel 332, thereby pushing panel 332 toward panel 330.

As shown in FIG. 4, panel 330 is positioned against the support wall 326 of base 304, such that panels 330 and 332 are aligned opposite one another. As shown in FIGS. 5 and 8A, panel 330 also includes an opening 349, for receiving sliding pad 346. Similarly, as shown also in FIG. 8B, panel 332 includes an opening 348, for receiving sliding pad 344. As described below, sliding pads 344 and 346 are adapted to engage a wire 102 positioned between the two pads.

The drive system 310 also operates sliding pads 344 and 346, as shown in FIG. 5. The pads 344 and 346 are moveable within openings 348 and 349, respectively, in a longitudinal direction. When the panels 330 and 332 are pressed against each other by rotation of the actuator 336, ridges 347 on pads 344 and 346 engage one another, thereby allowing longitudinal movement of pad 344 to result in longitudinal movement of engaged pad 346. It will be appreciated that the pads 344 and 346 can otherwise be connected to provide matching longitudinal movement when engaged.

To provide movement of the pads 344 and 346 with the actuator 336, a pin 350 may extend from the rear surface of the sliding pad 344 (i.e., the surface not facing pad 346) to translate the movement of actuator 336 via drive system 310 to slide sliding pad 344. Pin 350 may be integrally formed with sliding pad 344, or pin 350 may be a separate element. As shown in FIG. 8C, the pin 350 provided on the rear surface of the sliding pad 344 extends out of an opening on the rear side of the panel 332 facing the actuator 336. In the embodiment shown, the panel 332 is provided with a rear plate 356. Rear plate 356 includes an elongate opening or track 358 through which pin 350 extends.

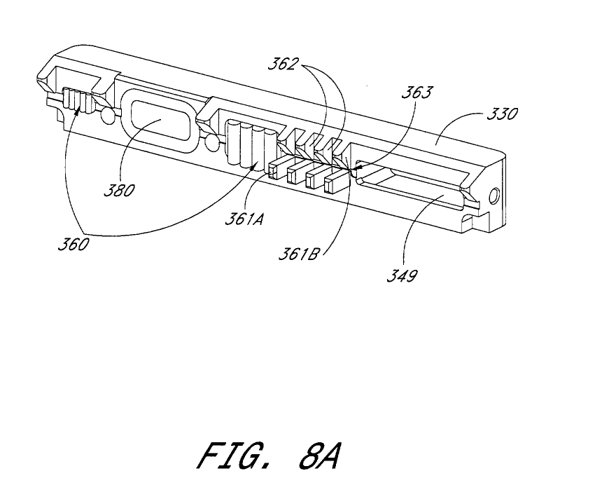

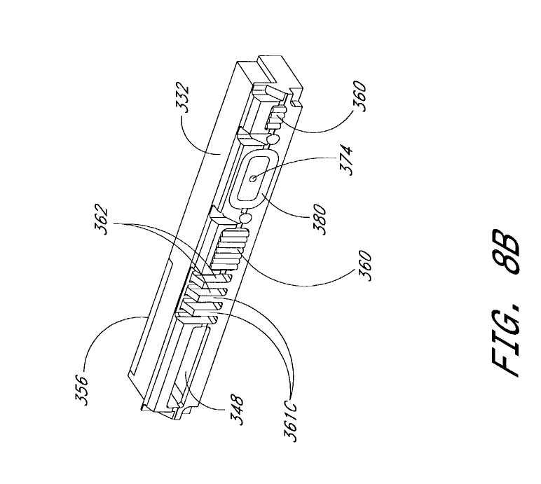

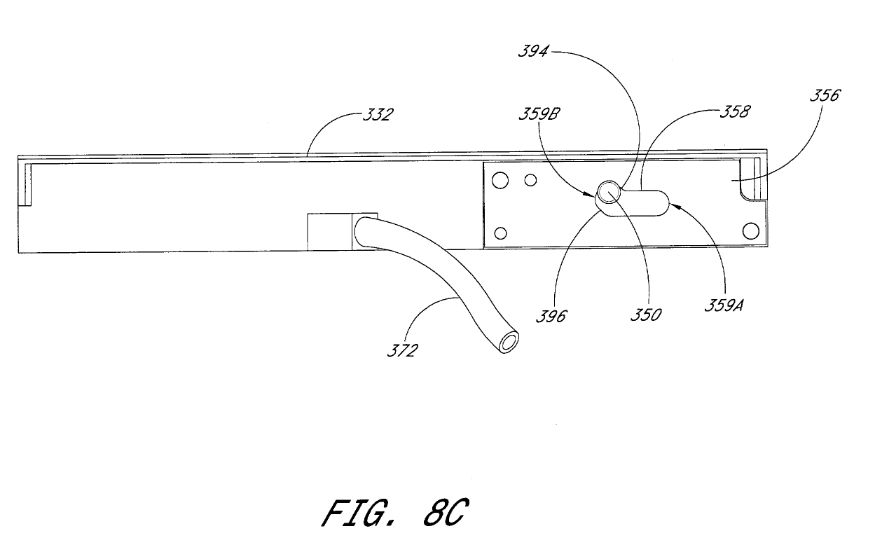

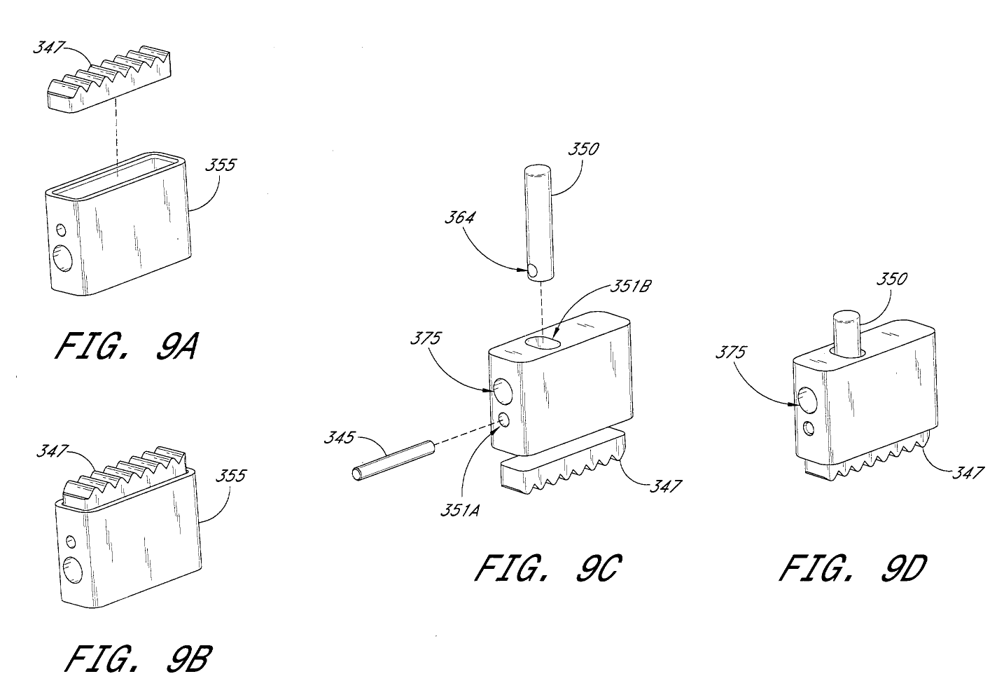

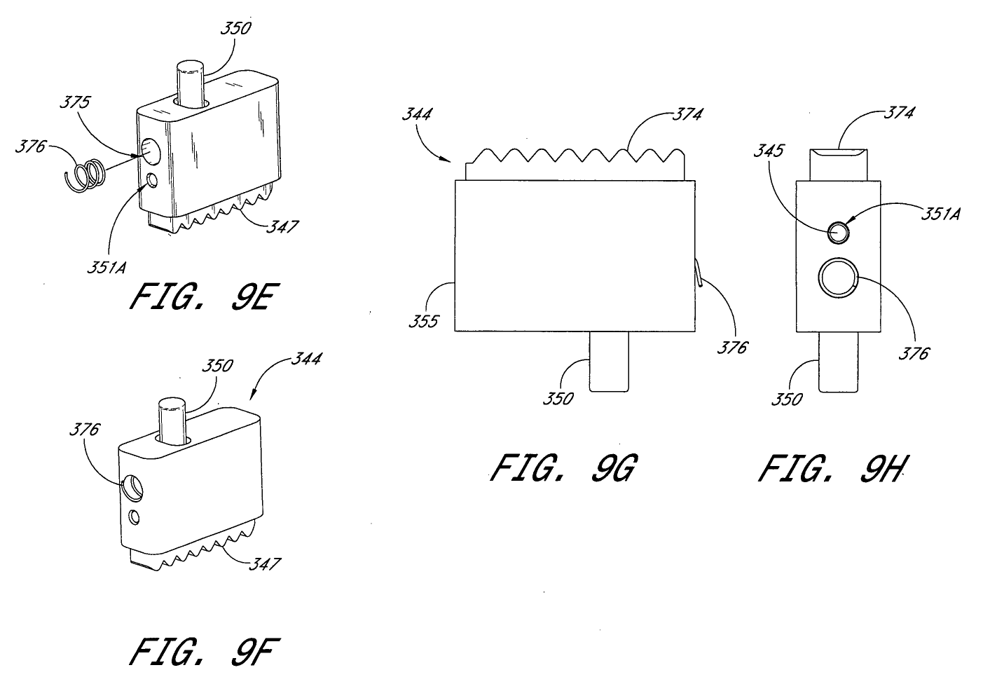

FIGS. 9A-9H illustrate one possible assembly sequence for the sliding pad 344. As shown in FIGS. 9A and 9B, ridges 347 are first attached to a rectangular block 355. As shown in FIG. 9C, rectangular block 355 includes a longitudinal channel 351A extending through the rectangular block 355, and a transverse channel 351B extending from the rear surface of the rectangular block 355 and intersecting the longitudinal channel 351A inside the rectangular block 355. Pin 350 is inserted into transverse channel 351B, as shown in FIG. 9D, and a rod 345 is inserted into longitudinal channel 351A, through a hole 364 in pin 350. As the channel 351B in the embodiment shown is sized to be larger than the pin 350, the pin 350 can rotate relative to the rod 345 to move vertically within channel 351B.

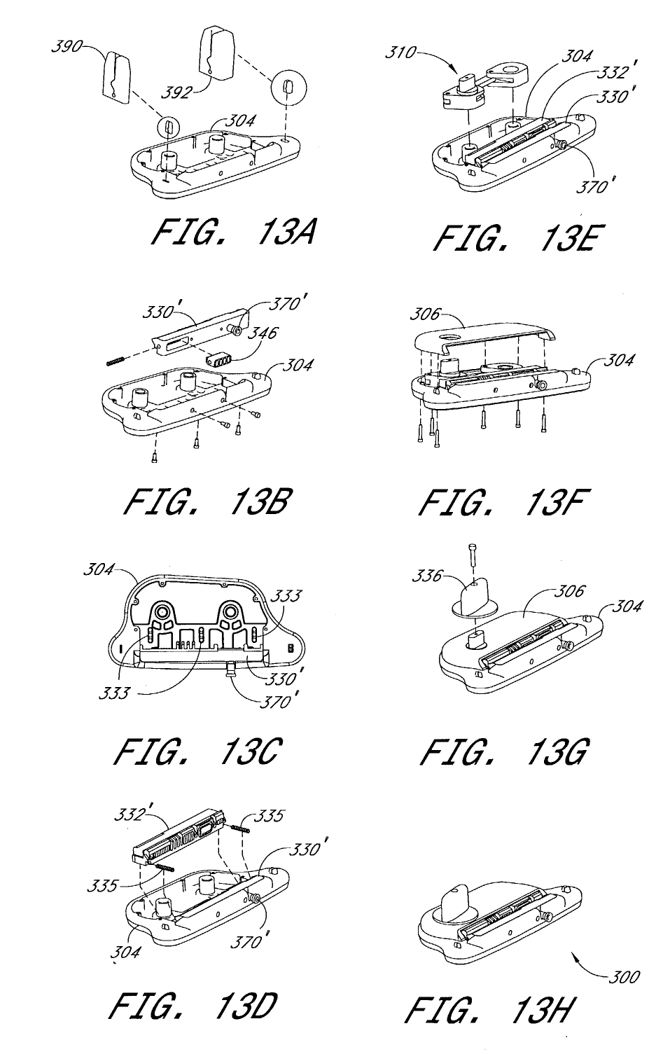

As shown in FIG. 9E, an opening 375 is provided at the distal end of the block 355, rearward of channel 351A. As shown in FIGS. 9E-9H, an offset spring 376 in inserted into opening 375. The spring 376 holds the pad 344 a short distance away from the most distal side of opening 348 when the pad 344 is inserted into the opening 348. Further details regarding this spring are described with respect to FIGS. 13A and 13B below.

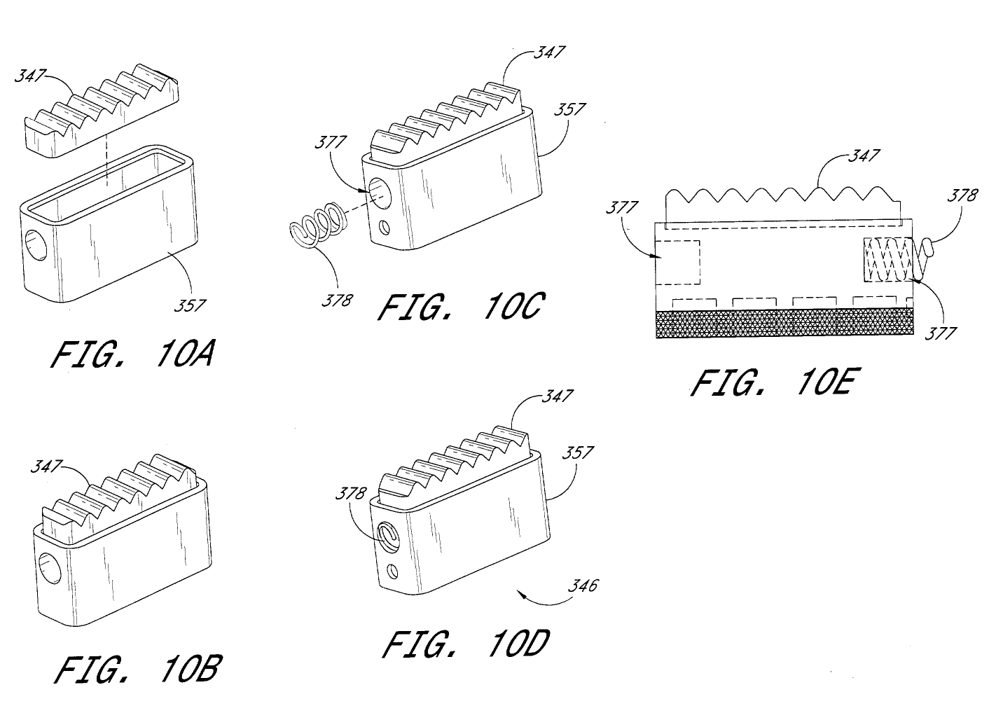

FIGS. 10A-10E illustrate an assembly sequence for pad 346. As shown in FIGS. 10A and 10B, like pad 344, the pad 346 comprises ridges 347 connected to a rectangular block 357. An opening 377 in the distal side of block 357, as shown in FIGS. 10C-10E, receives an offset spring 378, described further below. A proximal opening 379, shown in FIG. 10E in the proximal side of block 357, may also be provided to receive a return spring (not shown) used to bias the pad 346 distally within opening 349. It will be appreciated that a similar opening and return spring may be provided for pad 344.

As shown in FIGS. 5 and 7, a track 352 is provided on cam 338 to receive pin 350. Track 352 has a generally elongate shape with a proximal end 353A and a distal end 353B, and also includes a vertical slot 354 at distal end 353B. As the actuator 336 is initially turned, the pin 350 is positioned at the proximal end 353A of track 352, and the elongate portion of track 352 slides over the pin 350, bringing the pin closer to the distal end 353B, and causing panels 330 and 332 to move toward each other. Through this initial movement, the pin 350 remains stationary. As the pin 350 reaches distal end 353B, the slot 354 engages the pin 350, and continued turning of the actuator 336 moves the pin 350 along the track 358 in rear plate 356 in a distal to proximal direction.

As shown in FIG. 8C, the pin 350 in track 358 is initially positioned at distal end 359B as the actuator 336 is turned to clamp panels 330 and 332 together. Extending from the distal end 359B toward the proximal end 359A, the track 358 in one embodiment has a downwardly sloping ramp 394, such that as the pin 350 begins to move along track 358, it moves downwardly toward proximal end 359A. This downward movements forces the pin 350 down into the slot 354 of track 352 in cam 338. The movement of pin 350 along track 358 causes the movement of the pads 344 and 346 in a distal to proximal direction. Thus, when the pin 350 reaches the proximal end 359A of track 358, the pads 344 and 346 have moved from a distal position to a proximal position, which as described below, corresponds to the opening of valve mechanism 24 when guidewire 14 is inserted into the adaptor 300.

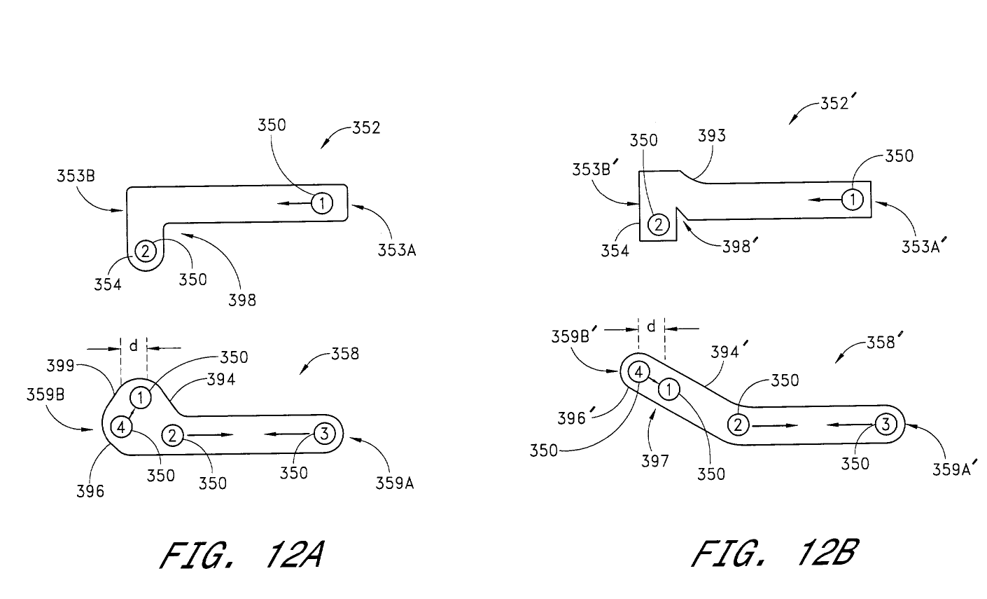

Rotating the actuator 336 in the reverse direction moves the pin 350, now positioned in the vertical slot 354, back along track 358 toward distal end 359B. Because the pin 350 is engaged in slot 354, the pin 350 cannot move along track 352 while it is moving along track 358. Toward the distal end 359B of track 358, an upwardly sloping ramp 396 moves the pin upward in the slot 354. Once the pin 350 reaches the distal end 359B, the pin 350 stops its movement, and the track 352 begins to slide over the pin 350. The track 352 slides over the pin 350 such that distal end 353B moves distally away from the pin 350 until the proximal end 353A of the track 352 is positioned over the pin 350. Further description and further embodiments of the relative movement of pin 350 within tracks 352 and 358 are provided below with respect to FIGS. 12A and 13B.

As shown in FIGS. 8A and 8B, panels 330 and 332 may each have textured surfaces 360, which in one embodiment can be a plurality of vertical grooves to facilitate the frictional engagement of panels 330 and 332 when a guidewire 14 and wire 102 are positioned within the adaptor 300. Distal to the openings 348 and 349, panels 330 and 332 each also include centering ridges 361A, 361B and 361C, with corresponding grooves 362, for receiving ridges of an opposing panel. More particularly, lower ridges 361A and upper ridges 361B on panel 330 as shown in FIG. 8A are designed such that when panel 332 slides toward panel 330, a guidewire 14 which is positioned between ridges 361A and 361B slides downward toward the panel 330 as guided by a sloped under surface of ridges 361B, within an channel 363 formed between the upper and lower ridges. The ridges 361C on panel 332 operate to push the guidewire 14 into the channel 363. These ridges help center the guidewire 14 and wire 102 across the faces of the panels 330 and 332 and prevent the guidewire 14 from bowing, especially when the wire 102 is moved.

As shown in FIG. 8B, a seal comprising gasket 380 is positioned around an opening 374 on panel 332, distal to the centering ridges 361C. A corresponding gasket 380 is provided on panel 330. Gaskets 380 are in alignment, such that when panels 330, 332 are brought together, a fluid tight inflation chamber is created within the interior region defined by gaskets 380. The fluid-tight inflation chamber is in fluid communication with fluid line 372 (see FIGS. 4 and 8C), so that a pressurized inflation fluid may be introduced into the fluid-tight inflation chamber by attaching an external pressurized fluid source to fluid line 372. Moreover, gaskets 380 may be formed of resilient materials, such as silicone, C-Flex (TM) and Pebax (TM), so that gaskets 380 may form-fit over a guidewire 14 tubular body which extends across the lateral edges of gaskets 380, to create the fluid-tight chamber.

As shown in FIG. 11, a fitting 370 is positioned on base 304, to act as a hub for fluid line 372 which terminates in opening 374 at panel 332. Fluid line 372 may include a standard luer connector which may be attached to a variety of existing external pressurized fluid sources, although other types of fittings, such as tubing, quick connects, and Y-site connections, may be easily substituted for a luer fitting.

As shown in FIG. 3, proximal and distal securing clips 390 and 392 may be optionally provided outside housing 302 to generally ensure proper alignment of guidewire 14 within channel 334. When a guidewire 14 is placed in channel 334, inflation port 52 will lie within the fluid-tight inflation chamber created by gaskets 380, and wire 102, but not proximal end 46, will rest between panels 330 and 332. Such longitudinal alignment can be assisted using markers on the guidewire 14 and wire 102, as described below.

For ease of understanding, the operation of inflation adaptor 300 to inflate the balloon 12 of the guidewire 14 of FIGS. 1-2B will now be described. As shown in FIG. 11, an inflation device 22 is attached to the fluid line 372. One suitable inflation device is available from Medtronic AVE under the name EZ FLATOR™. However, it will be appreciated that any number of syringe assemblies may be suitably used with the inflation adaptor 300.

The inflation device 22 shown in FIG. 11 comprises a low-volume inflation syringe 26 and a high capacity or reservoir syringe 28 encased together in a housing 30. An inflation knob 36 is disposed on the outside of the housing 30. Indicia 38 are preferably located on the housing 30 adjacent the knob 36 so that a clinician using the device can monitor the precise volume of liquid delivered by the inflation syringe 22. As depicted, the indicia 38 preferably comprise numbers corresponding to the size and shape of the balloon used. When the knob 36 is rotated from the “DEFLATE” or “0:” position to the number corresponding to the balloon in use, the syringe assembly 22 delivers the fluid volume associated with that balloon size. Alternatively, the indicia 38 could indicate the standard or metric volume of fluid delivered at each position. A deflation handle 40 is formed at a proximal end of the plunger 42. Preferably, the handle 40 is large, as illustrated in FIG. 11, and is easily held in a clinician's hand. Further details are described in U.S. Pat. No. 6,234,996, the entirety of which is incorporated herein by reference.

As shown in FIG. 11, guidewire 14, with the balloon 12 deflated, is inserted into the inflation adaptor 300 at channel 334. As described previously, guidewire 14 has an inflation port 52 located near proximal end 46, and a wire 102 extending from proximal end 46. Guidewire 14, with the valve mechanism 24 in the closed position, is placed within channel 334 of adaptor 300, and guidewire 14 and wire 102 are placed within securing clips 392 and 390, respectively, such that when panels 330, 332 are clamped together, inflation port 52 will lie within the fluid-tight inflation chamber created by gaskets 380, and wire 102, but not proximal end 46, will rest between pads 344 and 346.

Indicia (not shown) may be provided on guidewire 14 and wire 102, which when aligned with indicia on inflation adaptor 300, result in alignment of inflation port 52 with the fluid tight inflation chamber of adaptor 300, and alignment of wire 102 with sliding pads 344 and 346, when guidewire 14 and wire 102 are inserted into channel 334. Indicia may take the form of markings, grooves or notches, or any other suitable means of aligning the guidewire 14 and the wire 102 with the inflation adaptor alignment indicia. In one embodiment, the gap between indicia on guidewire 14 and wire 102 is approximately equal to the space between clips 390 and 392, such that by placing indicia within clips 390 and 392, guidewire 14 and wire 102 are properly aligned within adaptor 300.

Indicia alternatively may be located solely on the guidewire tubular body 44 to facilitate correct alignment. For example, two visible markings may be placed on the guidewire 14 on either side of the inflation port 52. By inserting the guidewire 14 into base 304 so that both of these markings are placed within gasket 380, the inflation port 52 will be within the fluid-tight inflation chamber created by gaskets 380 when panels 330 and 332 are in contact.

With the inflation device 22 attached, the fluid line 372 may be flushed using diluted contrast until the contrast flows out of the opening 374 inside the seal area defined by gaskets 380. Air can be aspirated from the adaptor 300 by fully retracting the deflation handle 40 on the inflation device 22 for about 2 to 5 seconds, then slowly releasing the handle to neutral.

When wire 102 and inflation port 52 are properly aligned within adaptor 300, inflation port 52 lies within the fluid-tight inflation chamber to be created by gaskets 380, and wire 102 rests between sliding pads 344 and 346. Actuator 336 is moved from its closed or first position to a second position, so that panels 330 and 332 clamp the guidewire 14 therein. Actuator 336 may be rotated about 30-90 degrees, more preferably about 75 degrees, to clamp guidewire 14 within panels 330 and 332. Guidewire 14, and more particularly wire 102, are centered across panels 330 and 332 during this rotation using the centering ridges 361A-C and grooves 362 described above.

The drive system 310 and clamping system 312 are shown in detail in FIG. 6. In use, when actuator 336 is moved from a first position to a second position, panel 332 moves toward panel 330, clamping guidewire 14 within channel 334. In the embodiment shown, clamping is effected by turning actuator 336 in a clockwise direction. However, it is envisioned that the clamping can be effected by turning actuator 336 in a counterclockwise direction. When actuator 336 is moved from its second position to a third position, sliding pads 344 and 346 move in a proximal direction parallel to the channel 334, through engagement of pin 350 with vertical slot 354, such that wire 102 moves away from proximal end 46, allowing fluid passage through port 52. In certain embodiments, the actuator 336 is rotated about 5 to 30 degrees, more preferably about 15 degrees, from the second to third position to slide pads 344 and 346. The adaptor is designed such that the length of travel of pad 344 provides at least the minimum sufficient distance to position the sealer portion 100 in the open or closed position, as desired.

Movement of actuator 336 from the second position to the third position causes pads 344 and 346 to move parallel to channel 334, along a longitudinal axis parallel to the longitudinal axis of the guidewire 14, and away from opening 374. The motion of the actuator from the first to second to third positions may be continuous or performed in steps. Because wire 102 is firmly secured between pads 344 and 346, a longitudinal force directed away from proximal end 46 is applied to wire 102. The longitudinal force on wire 102 results in the wire being partially withdrawn from lumen 50, which causes sealer portion 100 on wire 102 to be moved to a position within lumen 50 which is proximal of inflation port 52, as shown in FIG. 2B. The movement of sealer portion 100 proximally of inflation port 52 opens the valve mechanism 24, by establishing an unrestricted fluid pathway between inflation port 52 and balloon 12.

The external pressurized fluid source may then be activated, as for example by pushing the plunger on a syringe or turning inflation dial 36 in the inflation device 22 of FIG. 11, such that pressurized fluid passes through fluid line 372 and opening 374 into the fluid tight inflation chamber. The pressurized fluid then passes through inflation port 52 and lumen 50, to inflate balloon 12.

Inflated balloon 12 may be maintained in the inflated state, in the absence of the pressurized fluid source, by closing the valve mechanism 24. This is accomplished by moving actuator 336 back from the third position to the second position. The pads 344 and 346 apply a longitudinal force to the wire 102, directed toward the proximal end 46, causing wire 102 to be further inserted into lumen 50. Consequently, sealer portion 100 is moved within lumen 50 from a position which is proximal to inflation port 52 to a position in lumen 50 which is distal to inflation port 52. The fluid tight seal created by sealer portion 100 retains the pressurized fluid within lumen 50 and balloon 12, thereby maintaining balloon 12 in the inflated state. The fluid source can then be deactivated, and rotation of the actuator 336 back to its first position moves panel 332 away from panel 330. The adaptor and external pressurized fluid source may then be removed. With the valve mechanism 24 closed, inflation adaptor 300 may be removed by removing guidewire 14 and wire 102 from channel 334, and the inflation dial 36 of the inflation device 22 can be returned to “0”. With the balloon 12 properly inflated, various therapy catheters can be delivered and/or exchanged over the guidewire 14.

After treatment is complete, the guidewire 14 can be reinserted into the adaptor 300. The fluid line 372 is flushed as described above until diluted contrast flows out of the inflation port inside the seal area between gaskets 380. The actuator 336 is turned clockwise again to clamp the guidewire 14 and open the valve mechanism 24. The balloon 12 is deflated by retracting the deflation handle 40. The actuator 336 is turned counterclockwise to unclamp the guidewire 14, and the guidewire 14 is removed. A treatment catheter, such as the aspiration catheter described above, may remain on the guidewire 14 while the adaptor 300 is attached and used to deflate the balloon 12. After the balloon is deflated and the guidewire 14 is removed from the adaptor 300, the treatment catheter may be removed from the guidewire 14, or both devices can be removed together.

The track 352 of cam 338 and track 358 of panel 332 may further include an overdrive system, examples of which are illustrated in FIGS. 12A and 13B. Overdrive system as used herein minimizes the slippage that can occur between the wire 102 and the sliding pads 344 and 346, which can cause the wire 102 to not be fully reinserted distally into the lumen 50 of guidewire 14 when the valve mechanism 24 is being closed. In particular, when the pin 350 is returned to its initial position within track 358 of panel 332, the overdrive system moves the pin 350 an additional distance distally to ensure that the pads 344 and 346, and correspondingly the wire 102, return to an appropriate starting position to ensure that the valve mechanism 24 is fully closed.

FIG. 12A is a schematic diagram of the overdrive system utilized in the embodiment depicted in the figures described above. In particular, FIG. 12A shows schematically the relative movement of the pin 350 in the track 352 of cam 338 (from the perspective of viewing the track 352 from the opposite side of cam 338), and also shows schematically the relative movement of the pin 350 in the track 358 of panel 332. It will be appreciated that the tracks shown in FIG. 12A, as well as in FIG. 12B below, are illustrative, and therefore the relative dimensions of the track 352 and track 358 are not necessarily to scale. As shown in FIG. 12A, the pin 350 is in its initial or “1” position (corresponding to the first position of the actuator 336 above) when it resides both at the proximal end 353A of track 352 and distal end 359B of track 358.

As the actuator 336 is turned from its first position to its second position as described above, the pin 350 moves relatively within track 352 toward distal end 353B and vertical slot 354, while remaining in place at distal end 359B of track 358. When the pin 350 reaches the distal end 353B, movement of the actuator 336 from its second position to its third position causes the pin 350 to move from its “1” position to its “2” position within track 358, following the downward sloping ramp 394 of track 358. This alsocauses the pin 350 to move downward in vertical slot 354 to position “2”. This downward movement of the pin 350 within track 358 is facilitated in one embodiment by the manner in which the pin is operably connected to the sliding pad 344, described above with respect to FIGS. 9A-9H. Next, the pin 350, while remaining stationary in vertical slot 354, moves within track 358 toward proximal end 359A until it reaches position “3”. As described above, the movement of pin 350 to position “3” corresponds with the movement of pads 344 and 346.

As the actuator is reversibly turned from its third position back to its first position, the pin 350 remains in vertical slot 354 in track 352, and follows track 358 back toward its distal end 359B. As shown in FIG. 12A, on its return path, the track 358 includes an upwardly sloping ramp 396 adapted to move the pin 350 upward and out of the vertical slot 354 of track 352. Moreover, this upwardly sloping ramp 396 desirably extends further distally of the initial pin position “1”. Thus, when the pin 350 moves in track 358 toward distal end 359B, the pin 350 is actually moved longitudinally beyond its starting position to position “4”, as illustrated by the distance “d” in FIG. 12A. This distance d is the overdrive distance which drives the wire 102 further distally into lumen 50 of guidewire 14. In one embodiment, the overdrive distance d is about 0.01 inches. As the pin 350 moves to position “4,” it will be appreciated that the corresponding movement of pads 344 and 346 causes compression of offset springs 376 and 378, respectively, described above.

As the pin 350 travels along ramp 396, it begins to move upwardly out of the vertical slot 354. As the pin in track 352 starts to round corner 398, the pin 350 in track 358 moves along upwardly sloping ramp 399 to move the pin 350 from the “4” position back to the “1” position in track 358. This movement from the “4” position back to the “1” position in track 358 may be assisted by the offset springs 376 and 378 naturally biasing the pads 344 and 346 back proximally. Once the pin 350 completely rounds the corner 398, the pin 350 has reached the “1” position in track 358, and then moves back relatively toward proximal end 353A of track 352.

FIG. 12B illustrates an alternative embodiment for the shape and configuration of the tracks in cam 338 and in panel 332 which provide for relative movement of pin 350, also incorporating an overdrive system. These tracks are designated in FIG. 12B as tracks 352′ and 358′. In this embodiment, the track 358′ includes an angled slot 397 sloping downward from its distal end 359B′ toward the proximal end 359A′, before straightening out into a substantially horizontal path. Thus, as the pin 350 moves from the “1” position to the “2” position by turning of actuator 336, the pin engages the vertical slot 354′ at distal end 353B′, and then the angled slot 397 directs the pin 350 downward into the vertical slot 354. Movement of the pin 350 in the track 358′ continues as described above to position “3”.

When it is desired to return the pin 350 to its initial position, the actuator 336 is turned to move the pin 350 back toward its distal end 359B′. As the pin 350 moves upwardly in angled slot 397 to allow the pin 350 to escape vertical slot 354′, it will be seen that a bump 398′ is provided above the longitudinal height of the horizontal portion of track 352′. To overcome this height, the pin 350 moves further upward in angled slot 397 to position “4”, which extends a horizontal distance “d” beyond the initial position “1” of the pin 350. This distance d represents the overdrive of the pin 350, corresponding to the further movement of the pads 344 and 346 to push the wire 102 farther distally into lumen 50. As the pin 350 moves out of the vertical slot 354 and moves past the bump 398′, the downwardly sloping ramp 393 lowers the pin 350 back into its “1” position within track 358′. As described above, offset springs may also be used to return the pin 350 to its “1” position in track 358′.

FIGS. 13A-13H illustrate one sequence for assembling an inflation adaptor similar to the adaptor 300 described in FIGS. 3-11 above. This adaptor has substantially identical components as the previously described adaptor, with the exception that fluid line 372 is no longer provided and connected to an opening 374 in panel 332. Rather, the opening 374 is provided in panel 330′ within gasket 380 (not shown), and is in fluid communication with a luer port 370′ that extends through the wall supporting the panel 330′. Thus, an inflation source can be connected to port 370′ to inflate a guidewire 14 placed within the adaptor 300.

As shown in FIG. 13A, the base 304 as illustrated and as described above is provided, and clips 390 and 392 are attached at proximal and distal ends of the base 304, respectively. As shown in FIG. 13B, the panel 330′, which includes sliding pad 346 and luer port 370′, is positioned in the base 304. Ball bearings are then placed in tracks 333 of the base 304, as shown in FIG. 13C. Next, as shown in FIG. 13D, panel 332′ is positioned in the base 304 across from panel 330′, with springs 335 placed in between the two panels. The drive assembly 310 is positioned over the cylindrical protrusions within the base 304 (shown in FIG. 13E), and the cover 306 is placed over the base 304 and attached thereto (shown in FIG. 13F). As shown in FIG. 13G, the actuator 336 is attached over the cover 306 to the drive assembly 310, to form the completed adaptor 300 shown in FIG. 13H.

FIGS. 14A-14G illustrate one assembly sequence for the panel 332′ shown in FIGS. 13A-13H. As shown in FIGS. 14A and 14B, the panel 332′ includes opening 348, and pins 365 positioned distal to opening 348 adapted to receive rear plate 356, described below. As shown in FIG. 14C, textured surfaces 360, which in the embodiment shown include a ridged mid-pad and ridged distal pad, are provided on opposite sides of gasket 380. Guide pins 366, as shown in FIG. 14D, are provided on opposite sides of gasket 380 between the gasket 380 and each of the textured surfaces 360. These guide pins 380 are designed to engage openings in panel 330′ (not shown) such that the panels 332′ can reliably slide toward and away from panel 330′.

FIG. 14E illustrates the sliding pad 344 being inserted into the opening 348. A return spring 368 is provided proximal of the sliding pad 344, and may be inserted into a proximal opening (not shown) in rectangular block 355. The return spring 368 biases the sliding pad 344 in a distal position. Rear plate 356 is attached to the rear surface of panel 332′, with pins 365 extending through openings in the rear plate 356 and using screws 367 to attach the rear plate 356 to the panel 332′. The completed panel 332′ is illustrated in FIGS. 14F and 14G.

B. Second Embodiment

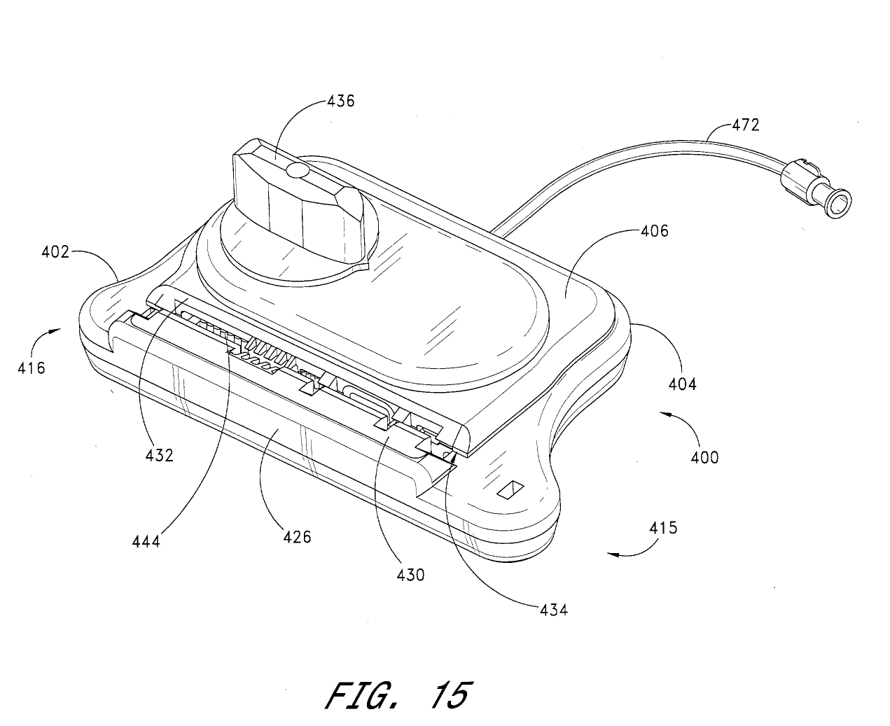

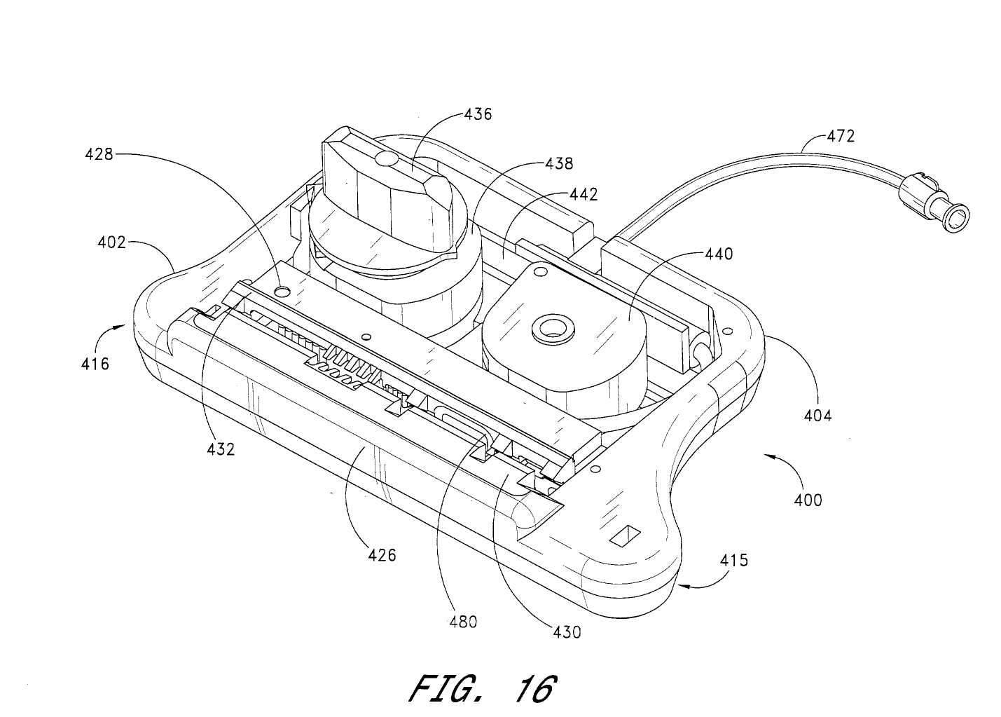

In accordance with another embodiment of the present invention, referring to FIGS. 15-25, there is illustrated an inflation adaptor which may be used to inflate and to open and close the valve mechanism 24 depicted in FIGS. 2A-2B. With reference to FIGS. 15-16, inflation adaptor 400 comprises a housing 402. Adaptor 400 integrates certain inflation components of an inflation device, such as assembly 22 of FIG. 11, within housing 402. Therefore, fewer components are required for operation and inflation of the balloon, thereby simplifying the procedure.

Where appropriate, like components between the second embodiment adaptor and the first embodiment adaptor will utilize corresponding reference numerals, with the reference numerals of the second embodiment adding 100 to the corresponding reference numerals of the first embodiment. It will therefore be appreciated that many principles of the construction and operation of the components of the first embodiment can be applied to the corresponding components of the second embodiment.

Housing 402 has a base 404 and a cover 406, which may be formed of metal, medical grade polycarbonate, or the like. However, as will be appreciated by those of skill in the art, a great many other materials may by used to form adaptor 400, including metals such as 300 series stainless steel and 400 series stainless steel, and polymeric materials such as acrylonitrile-butadiene-styrene (ABS), acrylics, and styrene-acrylonitriles. Furthermore, the base 404 and cover 406 may be manufactured in a variety of ways. For example, where polymeric materials are used, it is preferable to use a mold to manufacture the base 404 and the cover 406. Moreover, in some embodiments, more than one molded piece may be used to form base 404 and cover 406, with the various pieces being joined together by bonding or mechanical means to form either base 404 or cover 406. Alternatively, as is known in the art, base 404 and cover 406 can be formed through machining processes performed on larger blocks of the raw materials.

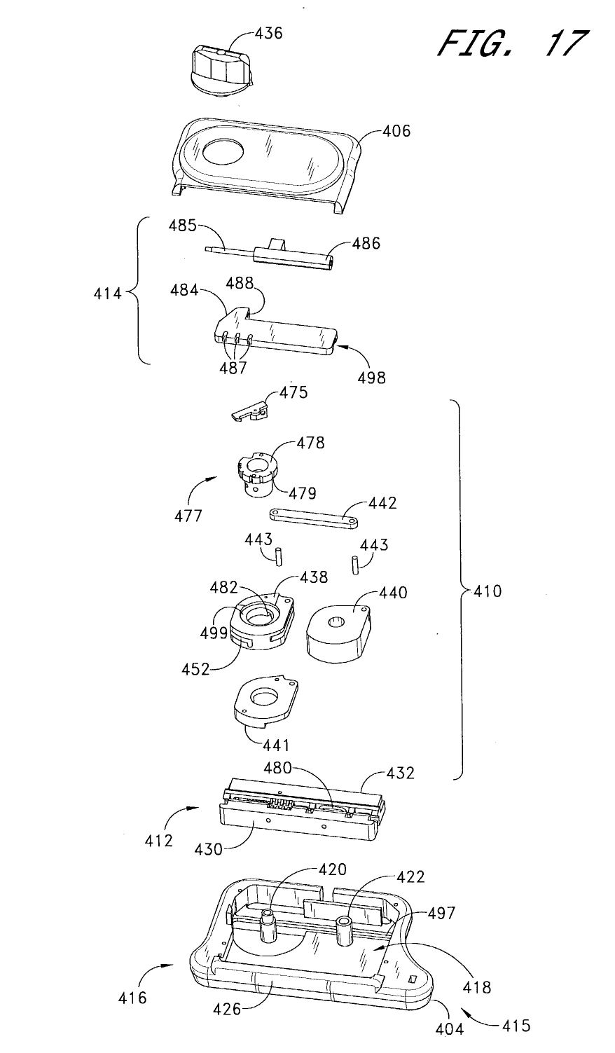

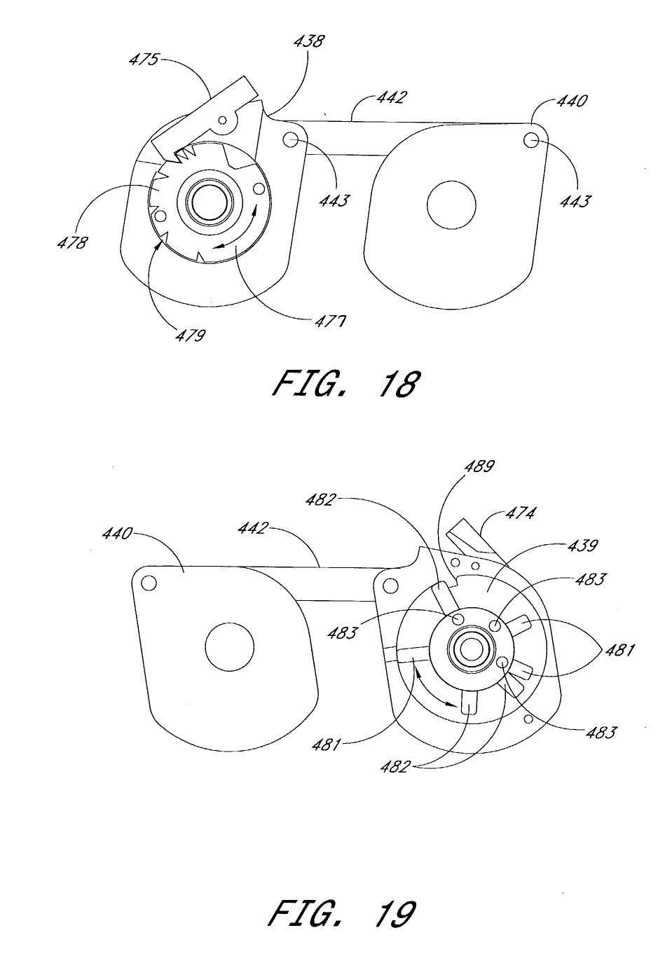

As shown in FIG. 17, adaptor 400 also includes a drive system 410, a clamping system 412, and a fluid delivery and inflation system 414, as will be described hereinafter. Cover 406 may be permanently fixed to base 404, enclosing the drive system 410, clamping system 412, and fluid delivery and inflation system 414 within housing 402. For example, a plurality of screws may secure the base 404 and cover 406 to one another. Alternatively, cover 406 may be releasably secured to base 404 by a pair of hinges positioned on one of the lateral edges of base 404 and cover 406, such that base 404 and cover 406 may be separated or joined in a clam shell manner.

As shown in FIG. 17, central recess 418 in base 404 includes a pair of cylindrical protrusions 420 and 422, for supporting the drive system 410. The base 404 includes a horizontal wall 426 for supporting panel 430, as described below. The base 404 as illustrated has a proximal end 415 and a distal end 416.

Panel 430 is substantially affixed against the support wall 426. A panel 432 in one embodiment is positioned across from panel 430, and is moveable within recess 418 toward and away from panel 430 such as described with respect to the first embodiment above. Recess 418 may also include a track with ball bearings and/or guide pins (not shown) to guide the movement of panel 432. Channel 434 is defined between panels 430 and 432 and is configured to receive guidewire 14. The panels 430 and 432 may be constructed and designed in a similar manner to the panels 330 and 332 described above. In particular, as shown in FIGS. 15 and 21, both panels 430 and 432 include sliding pads 446 and 444, respectively, which are comparable to pads 346 and 344 shown in the first embodiment above. As shown in FIG. 21, the sliding pad of panel 432 may be connected to a pin 450 adapted to engage track 452 on cam 438 in a similar manner to the first embodiment described above.

An actuator 436 is positioned on the external surface of cover 406, as shown in FIG. 15. The actuator may be a knob, which may be turned about 30-360 degrees, more preferably about 90-180 degrees. Actuator 436 in one embodiment is turned in a clockwise direction, but may also be adapted to be turned in a counterclockwise direction.

In the embodiment illustrated in FIG. 17, actuator 436 controls and is operable connected to cams 438 and 440, connected by a link 442 through pins 443, to form drive system 410. The lobe shape of cams 438 and 440 applies a force upon contact to panel 432 when the actuator 436 is turned, thereby sliding panel 432 toward panel 430, such as described with respect to the first embodiment above.

More particularly, as shown in FIG. 17, cam 438 in one embodiment is hollow and includes a shelf 499 for receiving a cylindrical gear member 477 which extends within cam 438. Cylindrical gear member 477 includes a ratchet 478 which is adapted to seat against the shelf 499. The cylindrical gear member 477 may be connected and attached to the actuator 436, for example using pins, such that turning of the actuator will also turn the cylindrical gear member 477. A support plate 441 is attached to the underside of cam 438, with a lower portion of cylindrical gear member 477 extending through a hole in support plate 441.

FIG. 18 illustrates a top view of a subassembly including cams 438 and 440 connected by link 442, with cylindrical gear member 477 inserted into cam 438 and a pawl 475 attached to the cam 438 and adapted to engage notches 479 in ratchet 478, as described below. FIG. 19 illustrates a bottom view of the subassembly, with the plate 441 removed. The cylindrical gear member 477 includes a plurality of radially extending protrusions 481 and downwardly extending protrusions 483. Protrusions 481 act as keys to engage slots 482 in a bottom surface of cam 438, such that when the protrusions 481 are aligned with slots 482, the cylindrical gear member 477 can move upward into the cam 438. The downwardly extending protrusions 483 act as keys to engage slots 487 in sliding plate 484, described further below.

The pawl 475 illustrated in FIG. 18 may be hingedly fixed to the cam 438 and may be spring biased to cause pawl 475 to move against the ratchet 478 of the cylindrical gear member 477. When the cylindrical gear member 477 is in a down position, such that protrusions 481 are not aligned with slots 482, the cylindrical gear member 477 can rotate relative to the cam 438, with the protrusions 481 sliding along a track in the underside of the cam 438 beneath slots 482. The pawl 475, which is biased against the ratchet 478, is adapted to engage notches 479. These notches 479 may be angled to allow rotation of cylindrical member 477 only in one direction, e.g., the clockwise direction, but not in the reverse direction. As described below, the location of these notches 479 may be selected to correspond to a desired amount of travel of the actuator 436, which in turn corresponds to a desired amount of fluid delivered through the fluid delivery system 414. It will be appreciated that to release the pawl 475 from a notch 479 to turn the cylindrical member 477 in the reverse direction, the pawl 475 should be pivoted to move away from cylindrical gear member 477.

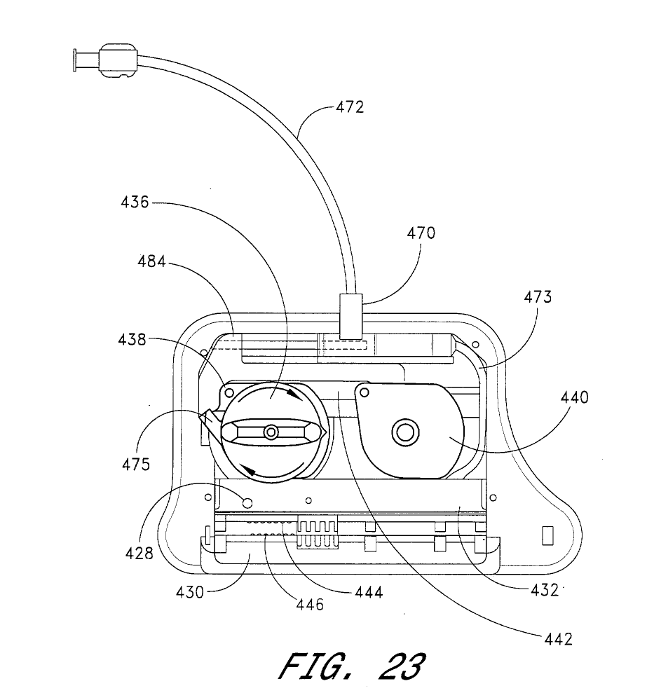

Also provided within the recess shown in FIG. 17 is the fluid delivery and inflation system 414 (shown also in a top view in FIG. 23). This system includes a sliding plate 484 with a plurality of spaced grooves or slots 487. The plate 484 slides longitudinally along a protruding track 497 provided within recess 418 extending proximally to distally. The sliding plate 484 includes an underside groove 498 for receiving the track 497. The sliding plate 484 also includes a proximal extension 488 attached to a plunger 485 which extends distally away from the proximal extension. The plunger 485 extends into an inflation barrel or cylinder 486 which is in fluid communication with a fluid line 472, shown in FIG. 21. Movement of the plunger in a proximal to distal direction pushes fluid contained in the cylinder 486 through a fluid line 473 to opening 474 in panel 432.

Movement of the plate 484 may be controlled with the actuator 436. More preferably, as shown in FIG. 19, the cylindrical gear member 477 to which actuator 436 is connected includes protrusions 483, for example three protrusions, which are adapted to engage slots 487. When cylindrical gear member 477 is moved relative to the cam 438, the protrusions 483 progressively engage the slots 487, thereby causing the sliding plate 484 to move in a proximal to distal direction. This movement causes plunger 485 to move within cylinder 486 to inflate a balloon 12 on a guidewire 14, described below.

For ease of understanding, the operation of inflation adaptor 400 to inflate the balloon 12 of the guidewire 14 of FIGS. 1-2B will now be described. The adaptor 400 may be connected to a fluid source, such as a syringe, through fluid line 472. The adaptor 400 can be prepped in a manner similar to the preparation of adaptor 300 above by flushing diluted contrast through the fluid lines 472 and 473 to opening 474. Guidewire 14, with the balloon 12 deflated, is inserted into the inflation adaptor at channel 434. As described previously, guidewire 14 has an inflation port 52 located near proximal end 46, and a wire 102 extending from proximal end 46. Guidewire 14, with the valve mechanism 24 in the closed position, is placed within channel 434 of adaptor 400, and guidewire 14 and wire 102 are placed within securing clips (not shown), if provided. This allows the inflation port 52 to lie within the fluid-tight inflation chamber created by gaskets 480, and the extending portion of wire 102, but not proximal end 46, to rest between pads 444 and 446.

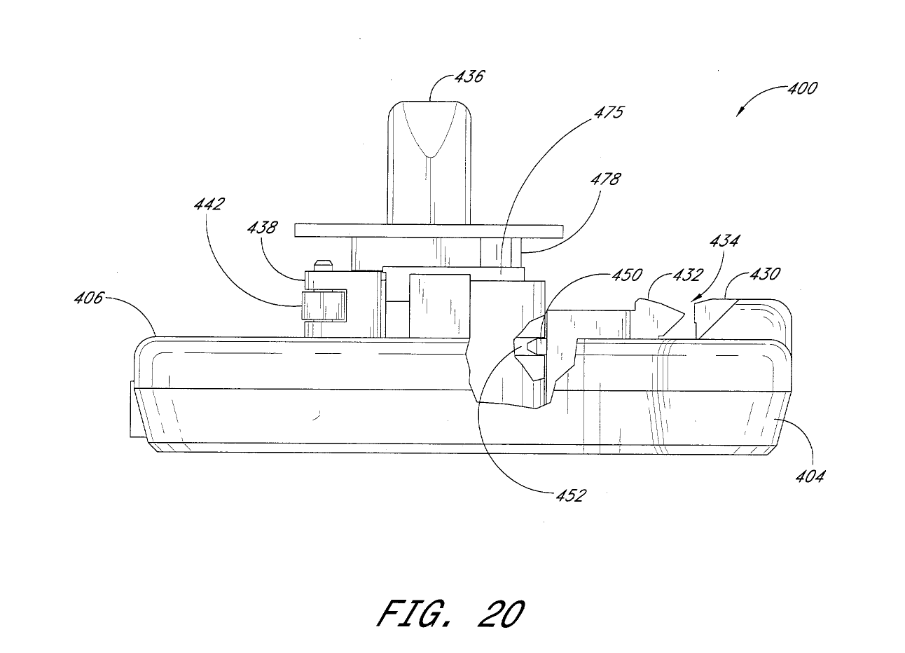

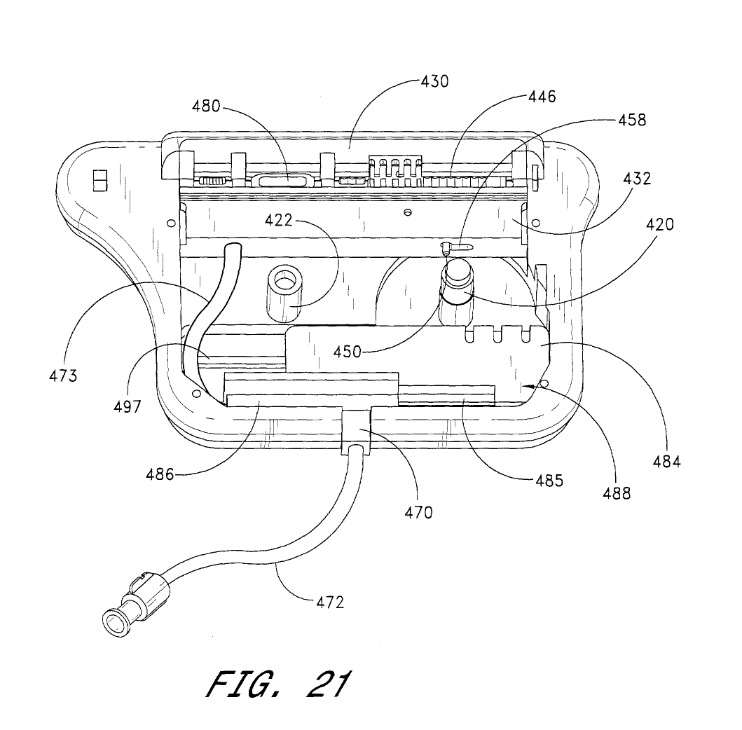

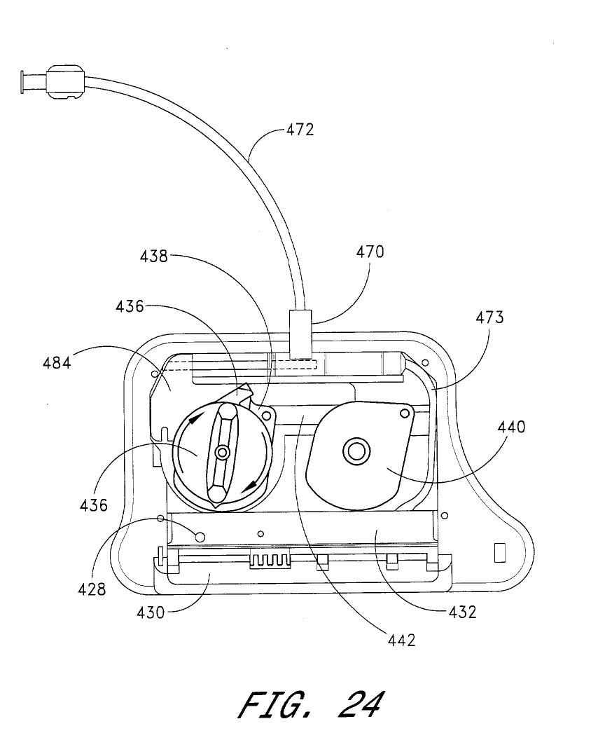

When wire 102 and inflation port 52 are properly aligned within adaptor 400, actuator 436 is in its first position, shown in FIGS. 20-23, wherein the panels 430 and 432 are spaced apart from each other. Actuator 436 when in its first position is in an up position, meaning that protrusions 481 of cylindrical gear member 477 engage slots 482 in cam 438. In one embodiment, the actuator 436 and cylindrical gear member 477 are spring biased to remain in an up position. The actuator 436 is turned, for example clockwise, from its first position to a second position, so that panels 430 and 432 clamp the guidewire 14 therein, shown in FIG. 24. In certain embodiments, actuator 436 is rotated about 30-90 degrees, more preferably about 75 to 90 degrees, to clamp guidewire 14 within panels 430 and 432.

As actuator 436 moves from its first position to its second position, the track 452 in cam 438 moves along pin 450 until it engages vertical slot 454, such as described above. Once the pin 450 engages vertical slot 454, continued rotation of actuator 436, for example about 5 to 30 degrees, more preferably about 15 degrees, from its second position to a third position causes the pin 450 to move in a distal to proximal direction, such that sliding pads 444 and 446 move proximally. This thereby causes wire 102 to move away from proximal end 46, allowing fluid passage through port 52.

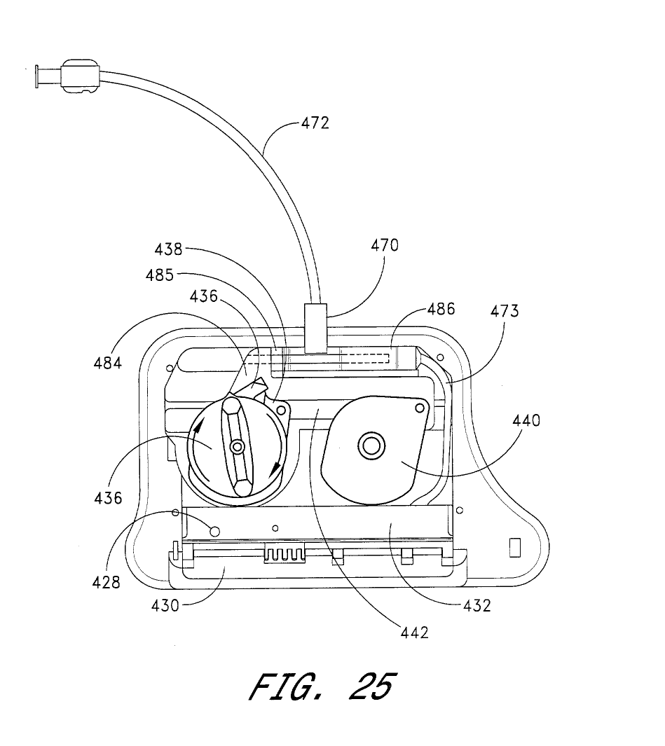

With the actuator 436 in its third position and the valve mechanism 24 open, the actuator 436 is pressed downward to disengage the cylindrical gear member 477 from the cam 438 and to engage the slots 487 of sliding panel 484 with protrusions 483 of cylindrical member 477. As shown in FIG. 25, the actuator 436 continues its clockwise rotation to a fourth position, and the protrusions 483 successively engage the spaced slots 487 to cause the sliding plate 484 to move distally. This distal movement causes the plunger 485 to move within cylinder 486, thereby displacing fluid inside the cylinder 486 into the balloon 12. The distance of travel of plunger 485 into cylinder 486 corresponds with a desired inflation volume of the balloon 12 on guidewire 14. Corresponding to this movement, the pawl 475 engages notches 479 in ratchet 478 as the actuator 436 is turned. The engagement of each successive notch 479 by pawl 475 corresponds with a predetermined distance of travel of the plunger 485 into cylinder 486. The actuator 436 can be turned until the pawl 475 is engaged with a notch 479, or until a protrusion 481 engages a stop 489 in the underside track of cam 438 (see FIG. 19).

After the balloon 12 has been inflated, the actuator 436, while still in its down position, can be turned in an opposite direction, e.g., counter-clockwise. If the pawl 475 is not already engaged with a notch 479, movement will continue unimpeded until pawl 475 engages a notch 479. Because of the angle of the notch 479, the cam 438 can no longer be rotated relative to the cylindrical gear member 477, and continued rotation of actuator 436 will turn cam 438 counter-clockwise, in turn engaging pin 450 in vertical slot 454 to push pin 450 distally to close the valve mechanism 24. The protrusions 483 do not re-engage the slots 487 of sliding plate 484 until the valve mechanism 24 is closed, such that the reverse rotation of the actuator 436 will not move the plunger 485 proximally within cylinder 486 until after the valve mechanism 24 is closed. As described above, an overdrive mechanism can be incorporated into the pin movement. Continued movement of the actuator 436 causes the cams to turn counter-clockwise to unclamp the panels 430 and 432, which may be spring biased to an open configuration. Once the panels 430 and 432 are separated, the guidewire 14 can be removed and various therapy or other catheters can be delivered and/or exchanged over the guidewire 14 to perform a desired treatment, as described above.

Once the treatment is completed, the guidewire 14 can be reinserted between the panels 430 and 432 and aligned as described above. The actuator 436 remains in its down position, and is turned clockwise causing relative movement between the cylindrical gear member 477 and cam 438 until a protrusion 483 again engages stop 489 as described above. Once this engagement is made, the cam 438 turns to clamp panels 430 and 432 together. Further rotation, as described above, moves the sliding pads 444 and 446 to open the valve mechanism 24. Once the valve mechanism 24 is opened, a syringe (not shown) attached to fluid line 472 can be used to draw fluid from the balloon 12 and thereby deflate the balloon 12. After deflation, the actuator 436 can be turned counter-clockwise once more as described above to close the valve mechanism 24 and unclamp the guidewire 14.

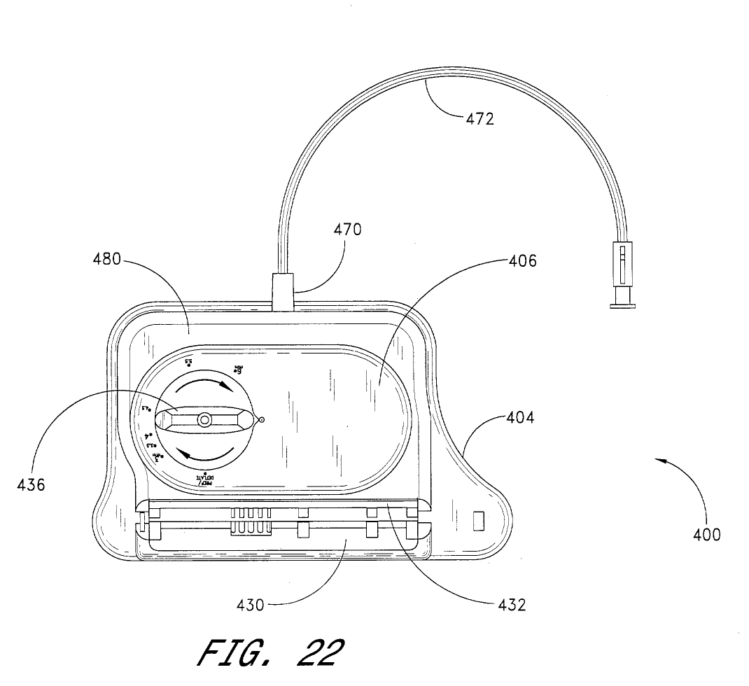

FIG. 22 illustrates one embodiment of the adaptor 400 showing a top cover 406 having indicia corresponding to the desired positions of the actuator 436. The actuator as shown is in its first or initial position, and turning of the actuator 436 clockwise to the “PREP/DEFLATE” position will move the actuator from its first position to its second position which clamps the guidewire 14 therewithin and continuously on to its third position wherein the valve mechanism 24 is open. In this position, the guidewire 14 can be prepped by drawing vacuum from the guidewire 14 and balloon 12. After the actuator 436 is pushed downward to disengage the cylindrical gear member 477 from cam 438, turning of the actuator 436 causes engagement of pawl 475 with notches 479, the location of the notches 479 corresponding with the indicia provided on the cover 406 and identifying a desired fourth position for the actuator. More particularly, the cover 406 in one embodiment illustrates sizes of 3 mm, 3.5 mm, 4 mm, 4.5 mm, 5.5 mm and 6 mm. These sizes represent desired diameters of the inflated balloon 12, with the spacing of the notches corresponding to the required distance the plunger 485 has to travel within cylinder 486 to cause appropriate fluid displacement to inflate the balloon 12 to these sizes.

A system for ensuring valve mechanism 24 is properly open may optionally be provided. In one embodiment, a color coded system may be used. In this embodiment, an opening 428 is formed through the upper surface of panel 432 or panel 430. As shown in FIG. 23, this opening 428 is provided in panel 432, which can only be seen through cover 406 when the panel 432 is clamped against panel 430. Sliding pad 444 within panel 432 may include a red and green sticker or paint on its upper surface which shows through the opening 428 in panel 432. The color-coded portion of sliding pad 444 coincides with the opening of panel 432, such that red shows when the valve mechanism 24 is closed, and green shows when the valve mechanism 24 is closed. Although the color-coded system has been described using red and green, it is envisioned that other colors may be used. Alternatively, the system for ensuring proper opening of the valve mechanism 24 may be audible. For example, a clicking noise may be heard to indicate when the valve mechanism 24 is open.

C. Third Embodiment

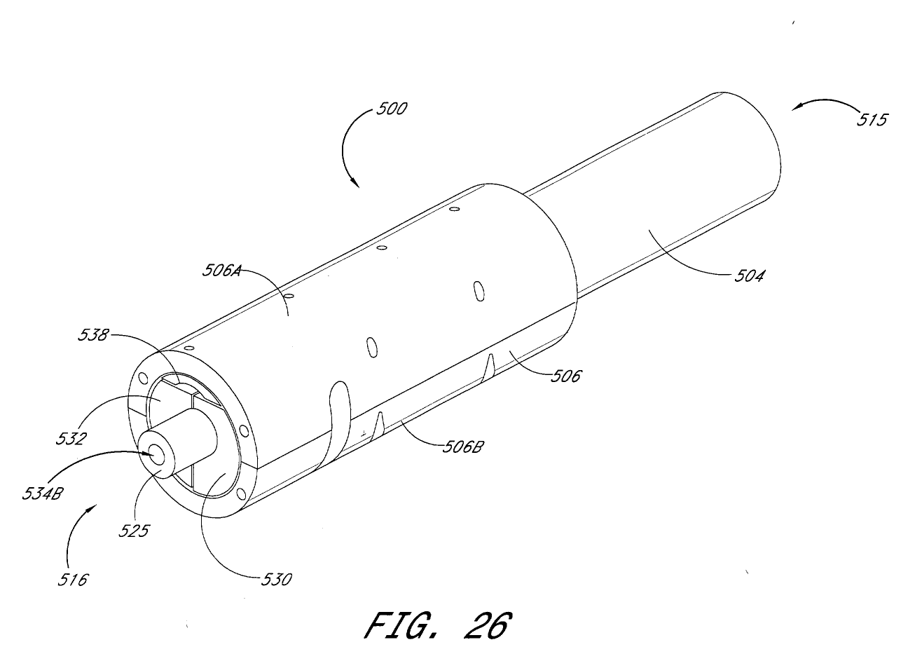

In accordance with another embodiment, an inflation adaptor 500 is shown in FIGS. 26-29 which may be used to inflate and to open and close the valve mechanism 24 depicted in FIGS. 2A-2B. Adaptor 500 as illustrated includes a base 504, which may be an elongate inner member extending between a proximal end 515 and a distal end 516. The proximal end 515 of the base 504 is cylindrical and forms a handle for grasping the adaptor 500. As described further below, toward distal end 516, the base 504 includes an integral panel 530, which operates in combination with panel 532 to manipulate an inner member relative to an outer tubular member, such as to inflate and open and close the valve mechanism 24 depicted in FIGS. 2A-2B.

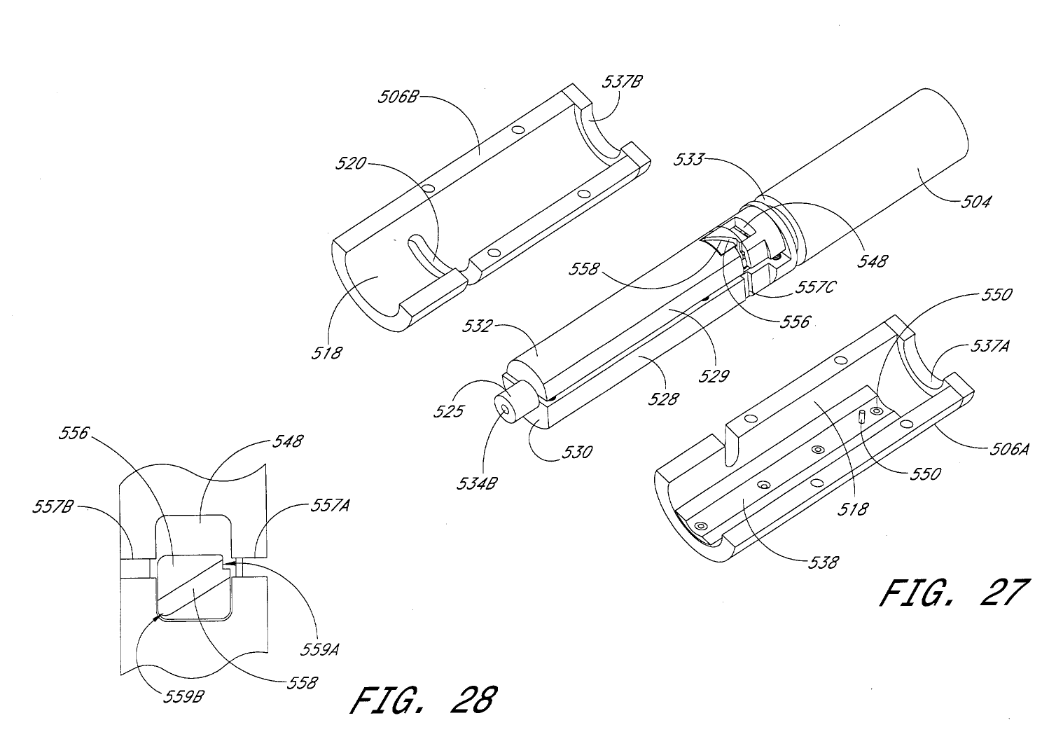

As shown further in FIG. 27, an outer shell 506 is provided over a distal portion of the base 504. The shell 506 has a cylindrical outer surface, and defines an interior surface 518 for receiving the base or inner member 504 with integral panel 530 and panel 532. In one embodiment, the outer shell 506 is divided into two halves 506A and 506B, which can be attached to each other by screws or other means.

The base 504 and outer shell 506 may be formed of metal, medical grade polycarbonate, or the like. However, as will be appreciated by those of skill in the art, a great many other materials may by used to form components of adaptor 500, including metals such as 300 series stainless steel and 400 series stainless steel, and polymeric materials such as acrylonitrile-butadiene-styrene (ABS), acrylics, and styrene-acrylonitriles. Furthermore, the base 504 and outer shell 506 may be manufactured in a variety of ways. For example, where polymeric materials are used, it is preferable to use a mold to manufacture the adaptor 500. Moreover, in some embodiments, more than one molded piece may be used to form outer shell 506, with the various pieces being joined together by bonding or mechanical means to form outer shell 506. Alternately, as is known in the art, outer shell 506 can be formed through machining processes performed on larger blocks of the raw materials. Base 504 and shell 506 may include a knurled surface for better gripping by the operator.

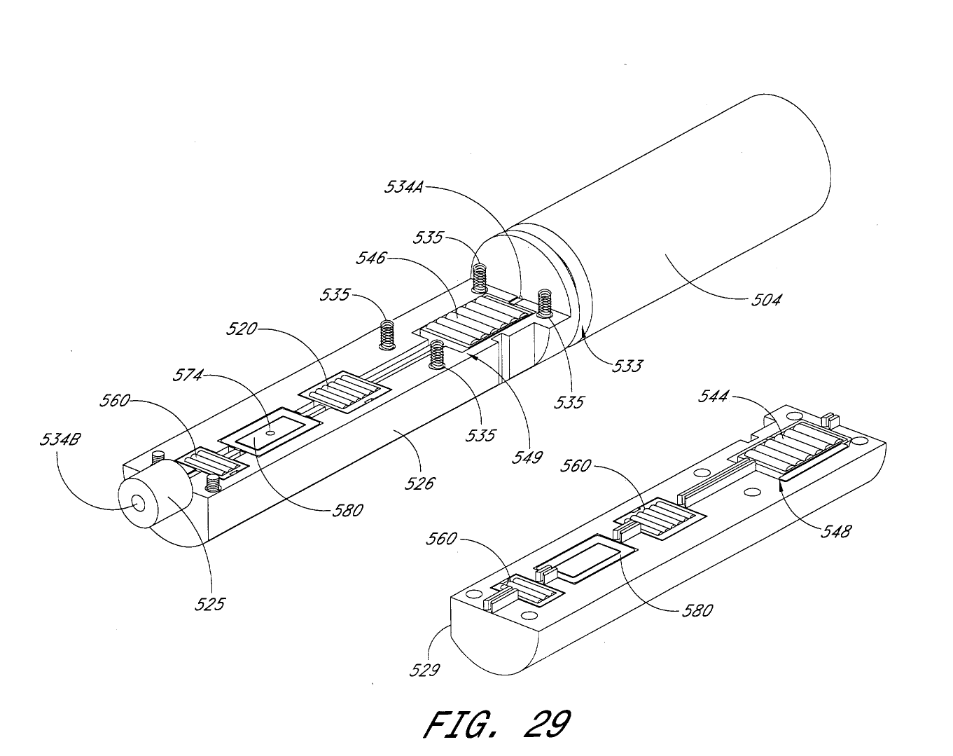

FIGS. 27 and 29 illustrate the adaptor 500 with the outer shell 506 removed. Within the shell 506 the base 504 includes panel 530, which may be integrally formed with the base 504. A corresponding panel 532 is provided over panel 530, with the panel 532 being moveable toward and away from panel 530. More particularly, springs 535 can be used to bias the panels apart. With the appropriate amount of force, as described below, panel 532 can be clamped against panel 530.

Panel 530 in one embodiment includes a cylindrical protrusion 525 at its distal end, which includes a channel 534B extending longitudinally therethrough for receiving guidewire 14. As illustrated in FIGS. 27 and 29, a proximal channel 534A extends through the proximal portion of base 504, such that a guidewire 14 can extend through the proximal portion of base 504 and out the proximal end, if desired.

As illustrated in FIG. 27, each panel 530 and 532 is substantially semi-cylindrical in shape. More preferably, each panel 530 and 532 includes a flat surface 528 and 529, respectively, extending along the length on one side of the panel. These flat surfaces correspond in location with a plate 538 that may be attached to and extends along the length of the interior surface of outer shell half 506A. With the exception of the location of the plate 538, the interior surface of shell 506 defines a substantially cylindrical inner surface. This cylindrical inner surface substantially mates with the cylindrical outer surfaces of the panels 530 and 532. However, because plate 538 protrudes into the volume defined within the outer shell 506, as shown in FIG. 26, when the shell 506 is assembled over the panels 530 and 532, the plate 538 abuts against the flat surfaces 528 and 529.

As shown in FIG. 29, the design of the panels 530 and 532, and in particular the design of the portions of the panels facing one another, are similar to the design of the panels described above with respect to the first embodiment. Thus, the panels 530 and 532 both include textured surfaces 560 and sealing gaskets 580. Moreover, panel 530 includes a fluid opening 574 which is in fluid communication with a fluid delivery device, described further below. As illustrated in FIG. 29, panel 530 includes a sliding pad 546, and panel 532 includes a sliding pad 544. As described with respect to pads 344 and 346 above, when pads 544 and 546 are pressed against one another, movement of pad 544 also causes movement of pad 546. Pads 544 and 546 are provided in openings 548 and 549, respectively, in panels 532 and 530, and are moveable longitudinally.

As shown in FIGS. 27 and 28, opening 548 in panel 532 extends through a rear surface of panel 532. A plate 556 which is connected to pad 544 is positioned in the opening 548, and is moveable longitudinally within the opening 548. Plate 556 includes a track 558 adapted to receive a pin 550, described below. In one embodiment, as illustrated, the track 558 extends diagonally across the plate. Also provided in the back surface of panel 532 are tracks 557A and 557B which are disposed on opposite sides of opening 548 extending in a direction perpendicular to the movement of plate 556. As illustrated, when the plate 556 is in its most distal position within opening 548, first end 559A of track 558 is directly adjacent track 557A. Correspondingly, when the plate 556 is in its most proximal position within opening 548, second end 559B of track 558 is directly adjacent track 557B.

Tracks 557A, 558 and 557B are adapted to receive a pin 550, which may be mounted to the interior surface of outer shell section 506A shown in FIG. 27, to cause movement of plate 556. In one embodiment, illustrated in FIG. 27, panel 530 also includes a track 557C extending in the same direction as track 557A and adapted to receive pin 550. More particularly, when the adaptor 500 is assembled, pin 550 protrudes into track 557A (or optionally, track 557C) to slide therein. The base 504 in one embodiment includes a circumferential track 533 for receiving protrusions 537A and 537B on the proximal interior surface of shell halves 506A and 506B, respectively. Pad 544 is initially located in its distalmost location. Then, when outer shell 506 is rotated counterclockwise over the panel 532 (as viewed from the distal side of the adaptor), the pin 550 follows track 557A to track 558. As rotation of outer shell 506 continues, pin 550 continues along track 558 from first end 559A to second end 559B. Sliding of the pin in this direction causes the plate 556 to slide proximally, thereby moving pads 544 and 546 proximally. Rotation of the shell can continue until pin 550 reaches the end of track 557B. Clockwise rotation of the shell 506 causes the pin 550 to move back along track 557B, into track 558 to move the pads 544 and 546 distally, and back into track 557A.

As illustrated in FIG. 26, the inner diameter of outer shell 506 varies due to the presence of plate 538. It will be appreciated that plate 538 need not be separate from shell 506, and thus can be formed integral therewith. When shell 506 is rotated counterclockwise relative to the panels 530 and 532, the plate 538 moves away from the flat surfaces 528 and 529 of the panels 530 and 532. The plate will therefore engage the back surface of panel 532, and as rotation continues, force the panel 532 against panel 530. Thus, as shell 506 is turned, the plate 538 clamps the panel 532 against panel 530. It will be appreciated that the inner surface of the plate 538 engaging the panel 532 need not be flat, and can be concave in shape to accommodate rotation of the plate around the panel 532.

As shown in FIG. 29, the panel 530 includes an opening 574 which allows fluid to flow into an inflation port 52 positioned between gaskets 580. The opening 574 is in fluid communication with a fluid line (not shown) extending from the back side of panel 530, through shell 506. More preferably, as shown in FIG. 27, cover 506 includes a slot 520 extending primarily through half 506B, which allows rotation of shell 506 without interfering with the fluid line.

The adaptor 500 of FIGS. 26-29 will now be described with respect to the guidewire 14 and wire 102 of FIGS. 1-2B above. The adaptor 500 is in its initial or first position when the plate 538 is adjacent the flat surfaces 528 and 529 of panels 530 and 532. In this position, the panels 530 and 532 may be spring biased away from each other to allow the guidewire 14 to pass into a channel defined there between. The wire 102 and proximal end of guidewire 14 may be inserted through channel 534B into the adaptor 500. Indicia or markings can be provided on guidewire 14 to align the guidewire 14 such that the inflation port 52 lies within the gaskets 580, and wire 102 lies between sliding pads 544 and 546. A fluid delivery source such as a syringe is connected to the fluid line (not shown) which is in fluid communication with opening 574.

It will be appreciated that other mechanisms can be provided for loading the guidewire 14 into adaptor 500. For example, the adaptor may be provided with a side access opening extending along the length of the adaptor. In such an embodiment, the shell 506 may be opened and closed in a clam-shell manner to clamp over the guidewire 14.

Once the guidewire 14 is aligned, the outer shell 506, acting as an actuator in a similar manner to the actuators 336 and 436 above, is turned, for example, counterclockwise (when the device is viewed from its distal end). Alternatively, the outer shell 506 can be held by an operator, for example with a left hand, while the proximal end of the base 504 is turned counter-clockwise with the right hand (when the device is viewed from its proximal end). The initial movement of the shell 502 causes the plate 538 to press against panel 532 to clamp the guidewire 14 between the panels 530 and 532. As rotation of the outer shell 506 continues, the pin 550 engages the tracks 557A, 558 and 557B as described above, causing the pads 544 and 546 to move distally to proximally. As the pads 544 and 546 engage the wire 102, this causes the wire 102 to move away from the proximal end of guidewire 14, and open a fluid passageway through inflation port 52. The longitudinal force on wire 102 results in the wire being partially withdrawn from lumen 50, which causes sealer portion 100 on wire 102 to be moved to a position within lumen 50 which is proximal of inflation port 52. The movement of sealer portion 100 proximally of inflation port 52 opens the valve mechanism 24, by establishing an unrestricted fluid pathway between inflation port 52 and balloon 12.

The external pressurized fluid source may then be activated, as for example by pushing the plunger on a syringe, such that pressurized fluid passes through the fluid line and opening 574 into the fluid tight inflation chamber. The pressurized fluid then passes through inflation port 52 and lumen 50, to inflate balloon 12.

Inflated balloon 12 may be maintained in the inflated state, in the absence of the pressurized fluid source, by closing the valve mechanism 24. By twisting of outer shell 506 or base 504 in the opposite direction, the fluid tight seal created by sealer portion 100 traps the pressurized fluid within lumen 50 and balloon 12, thereby maintaining balloon 12 in the inflated state. As the outer shell 506 returns to its initial position, the guidewire 14 is released from the clamping force of the panels 530 and 532, which now move apart. The external pressurized fluid source may then be deactivated and removed. Once the valve mechanism 24 is closed, inflation adaptor 500 may be removed by removing adaptor 500 from guidewire 14.

Although the present invention has been described in terms of certain embodiments, other embodiments of the invention including variations in dimensions, configuration and materials will be apparent to those of skill in the art in view of the disclosure herein. In addition, all features discussed in connection with any one embodiment herein can be readily adapted for use in other embodiments herein. The use of different terms or reference numerals for similar features in different embodiments does not imply differences other than those which may be expressly set forth. Accordingly, the present invention is intended to be described solely by reference to the appended claims, and not limited to the embodiments disclosed herein.

What is claimed is:

1. An adaptor for controlling actuation of an expandable device, the adaptor comprising:
a housing with a retaining portion which operates to releasably retain a section of a hollow tubular body therein, the expandable device being disposed at a distal end of the hollow tubular body; and
an actuator mounted on the housing, wherein movement of the actuator clamps the tubular body within the retaining portion of the housing and also moves a wire at least partially within the hollow tubular body from a first position to a second position, the movement of the wire between the first and second positions enabling actuation of the expandable device.

2. The adaptor of claim 1, wherein the retaining portion comprises a first panel and a second panel defining a channel there between for receiving said hollow tubular body.

3. The adaptor of claim 2, wherein the second panel is moveable toward the first panel to clamp the hollow tubular body there between.

4. The adaptor of claim 3, further comprising at least one cam, wherein movement of the actuator turns the cam to move the second panel toward the first panel.

5. The adaptor of claim 4, comprising a pair of cams connected by a link, wherein movement of the actuator turns said cams to move the second panel toward the first panel.

6. The adaptor of claim 1, wherein the actuator includes a rotatable knob.

7. The adaptor of claim 1, further comprising a pair of sliding pads adapted to engage said wire.

8. The adaptor of claim 7, wherein movement of the actuator is capable of moving said sliding pads in a longitudinal direction.

9. The adaptor of claim 1, wherein said expandable device is a balloon.

10. The adaptor of claim 9, further comprising a fluid line terminating in a fluid delivery opening within said retaining portion.

11. An adaptor for controlling actuation of an expandable device, the expandable device being disposed on a distal end of a hollow tubular body, the hollow tubular body surrounding a wire at least at a proximal end of the hollow tubular body, and wherein the wire is moveable within said hollow tubular body from a first position to a second position, the movement of the wire between the first and second positions enabling actuation of the expandable device, the adaptor comprising:
a housing having a first panel and a second panel disposed thereon, the first and second panels defining a channel there between, the channel being adapted to receive a portion of the hollow tubular body and the wire, said second panel being moveable toward and away from said first panel;
a first sliding pad and a second sliding pad positioned within openings of said first and second panels, respectively, and adapted to receive a portion of the wire there between, said sliding pads being cooperatively slidable within the openings of said first and second panels in a longitudinal direction; and
an actuator operatively connected to at least said second panel and at least said second sliding pad, wherein movement of said actuator causes said second panel to move relatively toward said first panel and also causes said sliding pads to move within the openings of the first panel and second panel;
wherein said actuator is moveable from a first position wherein the first panel and second panel are spaced apart, to a second position wherein the first panel and second panel are pressed against each other in order to clamp the hollow tubular body there between, and to a third position wherein the first sliding pad and second sliding pad move within the openings of the first and second panels to move the wire from its first position to its second position within said hollow tubular body.

12. The adaptor of claim 11, wherein the actuator is rotatable.

13. The adaptor of claim 11, wherein the actuator moves the second panel toward the first panel by turning at least one cam, said cam contacting said second panel to cause said second panel to move toward said first panel.

14. The adaptor of claim 13, further comprising a pin connected to the second sliding pad, said at least one cam engaging said pin when said cam is turned to move the second sliding pad in a distal to proximal direction.

15. The adaptor of claim 11, wherein said first and second sliding pads have a plurality of mating ridges and grooves, such that when said first and second sliding pads are pressed against each other, movement of said second sliding pad also causes movement of said first sliding pad.

16. The adaptor of claim 11, wherein said first and second panels include a plurality of mating ridges and grooves for centering the hollow tubular body across faces of said panels.

17. The adaptor of claim 11, wherein said expandable device is a balloon.

18. The adaptor of claim 17, wherein said first and second panels each includes a gasket adapted to engage one another to create a fluid-tight chamber which is in fluid communication with a fluid opening in one of said panels.

19. The adaptor of claim 11, wherein the housing includes a base and a cover adapted to be secured to one another.

20. The adaptor of claim 11, further comprising clips for securing the hollow tubular body within the adaptor.

21. An integrated inflation adaptor for inflating a balloon through an inflation port on a hollow tubular body, the adaptor comprising:
a housing with a retaining portion which operates to releasably retain a section of the hollow tubular body therein such that said inflation port is positioned within said retaining portion adjacent a fluid opening within said retaining portion, said retaining portion also adapted to retain a wire moveably disposed within a proximal end of said hollow tubular body; and
an actuator mounted on the housing, wherein the actuator is moveable from a first position to a second position to clamp the tubular body within the retaining portion of the housing, and is moveable from the second position to a third position to move said wire within said hollow tubular body in a distal to proximal direction across said inflation port, thereby allowing fluid flow from said fluid opening through said port.

22. The adaptor of claim 21, wherein the retaining portion includes a first panel and a second panel positioned within said housing and defining a channel there between, the channel being adapted to receive a portion of the hollow tubular body and the wire, said second panel being moveable toward and away from said first panel, such that movement of the actuator from the first position to the second position moves the second panel toward the first panel.

23. The adaptor of claim 22, further comprising a first sliding pad and a second sliding pad positioned within openings of said first and second panels, respectively, and adapted to engage said wire, said sliding pads being cooperatively slidable within the openings of said first and second panels in a longitudinal direction, wherein movement of the actuator from the second position to the third position slides said first and second sliding pads in a distal to proximal direction to move said wire proximally of said inflation port.

24. The adaptor of claim 23, wherein at least one of the sliding pads is spring biased to retain said sliding pad in a distal location.

25. The adaptor of claim 21, wherein the adaptor is moveable from said third position to a fourth position to cause fluid to flow into said port to inflate said balloon.

26. The adaptor of claim 25, further comprising an inflation cylinder and a plunger received in said inflation cylinder, said inflation cylinder being in fluid communication with said fluid opening, and wherein movement of said actuator from the third position to the fourth position moves the plunger into the inflation cylinder to displace fluid into the inflation port to inflate the balloon.

27. A method of manipulating a wire within a lumen of a hollow tubular body, the method comprising:
positioning the hollow tubular body and wire within a retaining portion of an adaptor, the retaining portion including a first panel and a second panel defining a channel there between for positioning of at least the tubular body, and first and second pads adapted to position the wire there between;
moving an actuator on the adaptor from a first position to a second position, the movement of the actuator causing the first panel and second panel to move relatively toward each other to clamp at least the hollow tubular body there between, wherein when the hollow tubular body is clamped between the first panel and second panel, the first and second pads contact the wire; and
moving the actuator from the second position to a third position, the movement of the actuator causing movement of the first and second pads in a direction parallel to a longitudinal axis of the hollow tubular body to cause corresponding movement of the wire relative to the hollow tubular body.

28. The method of claim 27, wherein the actuator is moved from the first position to the second position and the second position to the third position in one continuous motion.

29. The method of claim 27, wherein the actuator is rotated.

30. The method of claim 27, wherein movement of the actuator from the second to third position causes the pads to move from a distal location to a proximal location and also causes movement of the wire in a distal to proximal direction within the hollow tubular body from a distal location to a proximal location.

31. The method of claim 30, wherein the hollow tubular body includes an inflatable balloon at a distal end thereof and an inflation port at a proximal end thereof, and the wire includes a sealer portion at a distal end thereof, and movement of the actuator from the second to third position causes movement of the sealer portion from a position distal to said inflation port to a position proximal to said inflation port.

32. The method of claim 31, wherein at least one of the panels includes a fluid opening, such that when the panels are clamped against the tubular body the fluid opening is in fluid communication with the inflation port.

33. The method of claim 32, further comprising after moving the actuator from the second position to the third position, moving the actuator from the third position to a fourth position, wherein movement of the actuator pushes a plunger within the adaptor into an inflation cylinder to displace fluid in said cylinder through said fluid opening and into said inflation port to inflate said balloon.

34. The method of claim 30, further comprising, after moving the actuator from the first position to the third position, moving the actuator back to its first position to move the pads back to their distal location and to unclamp the hollow tubular body.

35. The method of claim 34, wherein moving the actuator back to the first position moves the pads distally beyond their distal location, thereby overdriving said wire within said hollow tubular body.

36. The method of claim 35, wherein the pads are spring-biased to return to their distal location after the actuator moves the pads distally beyond their distal location.
